(19) United States
(12) Patent Application Publication
(10) Pub. No.: US 2026/0308593 A1
(43) Pub. Date: Oct. 8, 2026

(54) METHODS FOR MITIGATING CARDIAC AGING AND STRESS AND IMPROVIING CARDIAC FUNCTION

(71) Applicant: San Diego State University (SDSU) Foundation, dba San Diego State University Research Foundation, San Diego, CA (US)

(72) Inventor: Mark SUSSMAN, San Diego, CA (US)

(21) Appl. No.: 19/100,861

(22) PCT Filed: Jul. 25, 2023
(86) PCT No.: PCT/US2023/028560

§ 371 Date: Feb. 3, 2025

Related U.S. Application Data
(60) Provisional application No. 63/396,553, filed on Aug. 9, 2022.

(51) Int. Cl. C12N 15/113; A61K 31/7105; A61K 45/06; A61K 47/68; C07K 16/18

(52) U.S. Cl. CPC C12N 15/1135; A61K 31/7105; A61K 45/06; A61K 47/6835; A61K 47/6889; C07K 16/18; C12N 2310/14

(57) ABSTRACT

Provided are methods for: removing cardiac cells impaired or functionally diminished by stress or age, wherein optionally cardiac cells impaired or functionally diminished have reduced proliferative capacity and/or polyploidy, initiating, inducing or accelerating a cardiac muscle repair or tissue regeneration, a cardiac vasculature repair or tissue regeneration or a cardiac connective tissue repair or tissue regeneration, treating or ameliorating age-related or stress-related cardiomyopathy, and/or treating or ameliorating a heart injury, an injury subsequent to (or following, or optionally from 1 minute to 12 hours after, or immediately after) a myocardial infarction (MI), comprising administering to an individual in need thereof a composition or treatment that ablates or inactivates Lin28+ cardiac cells, including Lin28+ cardiac stem cells or cardiac progenitor cells, comprising administering to an individual in need thereof a composition or treatment that ablates or inactivates Lin28+ cardiac cells, including Lin28+ cardiac stem cells or cardiac progenitor cells.

### RELATED APPLICATIONS

This Patent Convention Treaty (PCT) International Application claims the benefit of priority under 35 U.S.C. § 119(e) of U.S. Provisional Patent Application Ser. No. 63/396,553, Aug. 9, 2022. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under grant numbers R01HL067245 and R01HL135661 awarded by the National Institutes of Health. The government has certain rights in the invention.

### TECHNICAL FIELD

This invention generally relates to cell and molecular biology, cardiology and regenerative medicine. In alternative embodiments, provided are methods for: removing cardiac cells impaired or functionally diminished by stress or age, wherein optionally cardiac cells impaired or functionally diminished have reduced proliferative capacity and/or polyploidy, initiating, inducing or accelerating a cardiac muscle repair or tissue regeneration, a cardiac vasculature repair or tissue regeneration or a cardiac connective tissue repair or tissue regeneration, treating or ameliorating age-related or stress-related cardiomyopathy, and/or treating or ameliorating a heart injury, an injury subsequent to (or following, or optionally from 1 minute to 12 hours after, or immediately after) a myocardial infarction (MI), comprising: administering to an individual in need thereof a composition or treatment that ablates or inactivates Lin28+ cardiac cells, including Lin28+ cardiac stem cells or cardiac progenitor cells, or administrating to a patient a product of manufacture as provided herein.

### BACKGROUND

Lin-28 homolog A is a protein that in humans is encoded by the LIN28 gene. LIN28 encodes an RNA-binding protein that binds to and enhances the translation of the IGF-2 (insulin-like growth factor 2) mRNA. LIN28 is highly expressed in human embryonic stem cells.

Declining cellular functional capacity resulting from stress or aging is a primary contributor to impairment of myocardial performance. Molecular pathway regulation of biological processes in cardiac interstitial cells (CICs) is pivotal in stress and aging responses. Altered localization of the RNA binding protein Lin28A has been reported in response to environmental stress, but the role of Lin28A in response to stress in CICs has not been explored.

### SUMMARY

In alternative embodiments, provided are methods for:
removing cardiac cells impaired or functionally diminished by stress or age, wherein optionally cardiac cells impaired or functionally diminished have reduced proliferative capacity and/or polyploidy,
initiating, inducing or accelerating a cardiac muscle repair or tissue regeneration, a cardiac vasculature repair or tissue regeneration or a cardiac connective tissue repair or tissue regeneration,
treating or ameliorating age-related or stress-related cardiomyopathy, and/or
treating or ameliorating a heart injury or dysfunction, an injury subsequent to (or following, or optionally from 1 minute to 12 hours after, or immediately after) a myocardial infarction (MI),
comprising:
(a) (i) providing: a compound or composition capable of inhibiting or decreasing the activity of Lin28+, or ablating or killing a Lin28+-expressing cardiac cell,
wherein the compound or composition comprises:
   (1) a polypeptide composition capable of inhibiting or decreasing the activity of Lin28+, or ablating or killing a Lin28+-expressing cardiac cell, wherein optionally the polypeptide composition is or is comprised of an anti-Lin28+ antibody, and optionally the anti-Lin28+ antibody is a monoclonal antibody,
   (2) a nucleic acid composition that is capable of inhibiting or decreasing the activity of Lin28+, or ablating or killing a Lin28+-expressing cardiac cell; or
   (3) a compound capable of inhibiting or decreasing the activity of Lin28+, or ablating or killing a Lin28+-expressing cardiac cell,
and
(ii) introducing into, onto or approximate to (close to) the mammalian heart, or cardiac or heart tissue, or heart muscle, or cardiac vasculature or connective tissue: the compound or composition, or
(b) administering a pharmaceutically acceptable formulation or a pharmaceutical composition to a subject in need thereof,
wherein the pharmaceutically acceptable formulation or pharmaceutical composition is comprised of:
   (1) a polypeptide composition capable of inhibiting or decreasing the activity of Lin28+, or ablating or killing a Lin28+-expressing cardiac cell, wherein optionally the polypeptide composition is or is comprised of an anti-Lin28+ antibody or Lin28+ binding fragement thereof, and optionally the anti-Lin28+ antibody is a monoclonal antibody,
   (2) a nucleic acid composition that is capable of inhibiting or decreasing the activity of Lin28+, or ablating or killing a Lin28+-expressing cardiac cell; or
   (3) a compound capable of inhibiting or decreasing the activity of Lin28+, or ablating or killing a Lin28+-expressing cardiac cell.
thereby
removing cardiac cells impaired or functionally diminished by stress or age, wherein optionally the cardiac cells impaired or functionally diminished have reduced proliferative capacity and/or polyploidy,
initiating, inducing or accelerating cardiac muscle repair or tissue regeneration, cardiac vasculature repair or tissue regeneration or cardiac connective tissue repair or tissue regeneration,
treating or ameliorating age-related or stress-related cardiomyopathy, and/or
treating or ameliorating the heart injury or dysfunction, an injury subsequent to (or following, or optionally from 1 minute to 12 hours after, or immediately after) a myocardial infarction (MI).

In alternative embodiments, of methods as provided herein:
the anti-Lin28+ antibody carries a cytotoxic payload, wherein optionally the cytotoxic payload comprises a cytotoxic compound or cytotoxic radionuclide, and optionally the cytotoxic compound comprises calicheamicin, duocarymycin or a ribosome-inactivating protein (RIP), optionally ricin, and optionally the radionuclide comprises astatine-211, yttrium-90, lutetium-177 or iodine-131, and optionally the cytotoxic payload is covalently linked to the anti-Lin28+ antibody, optionally using a linker (optionally a linker comprising disuccinimidyl suberate (DSS)) or the cytotoxic payload is conjugated to the anti-Lin28+ antibody using a metal chelator, optionally diethylenetriaminepentaacetic acid (DTPA) or 1,4,7,10-tetraazacyclododecane-1,4,7,10-tetraacetic acid (DOTA);
the nucleic acid composition that is capable of inhibiting or decreasing the activity of Lin28+, or ablating or killing a Lin28+-expressing cardiac cell is or comprises an inhibitory nucleic acid, wherein optionally the inhibitory nucleic acid comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of a Lin28+ protein, transcript and/or gene;
the compound capable of inhibiting or decreasing the activity of Lin28+, or ablating or killing a Lin28+-expressing cardiac cell comprises: a small molecule, a lipid, a saccharide or a polysaccharide;
the compound comprises: CL285032; TPEN, LI171, 6-hydroxy-DL-DOPA; or N-methyl-N-[3-(3-methyl[1,2,4]triazolo[4,3-b]pyridazin-6-yl)phenyl]acetamide; or, an enantiomer or stereoisomer thereof,
the polypeptide, the nucleic acid and/or the compound is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally;
wherein optionally the formulation or pharmaceutical composition is contained in or carried in a nanoparticle, a particle, a micelle or a liposome or lipoplex, a polymersome, a polyplex or a dendrimer;
wherein optionally the compound, polypeptide or the nucleic acid, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant; and/or
the nucleic acid comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a cardiac cell.

In alternative embodiments, provided are kits or products of manufacture (for example, implants) comprising a compound, nucleic acid or polypeptide, or a formulation or a pharmaceutical composition, as used in a method of any one of the preceding claims, and comprising instructions on practicing a method as provided herein.

In alternative embodiments, provided are uses of a compound, nucleic acid or polypeptide, or a formulation or a pharmaceutical composition, as used in a method as provided herein, in the manufacture of a medicament for:
removing cardiac cells impaired or functionally diminished by stress or age, wherein optionally cardiac cells impaired or functionally diminished have reduced proliferative capacity and/or polyploidy,
initiating, inducing or accelerating a cardiac muscle repair or tissue regeneration, a cardiac vasculature repair or tissue regeneration or a cardiac connective tissue repair or tissue regeneration,
treating or ameliorating age-related or stress-related cardiomyopathy, and/or
treating or ameliorating a heart injury or dysfunction, an injury subsequent to (or following, or optionally from 1 minute to 12 hours after, or immediately after) a myocardial infarction (MI).

In alternative embodiments, provided are compounds, nucleic acids or polypeptides, or formulations or pharmaceutical compositions, as used in a method as provided herein, for use in:
removing cardiac cells impaired or functionally diminished by stress or age, wherein optionally cardiac cells impaired or functionally diminished have reduced proliferative capacity and/or polyploidy,
initiating, inducing or accelerating a cardiac muscle repair or tissue regeneration, a cardiac vasculature repair or tissue regeneration or a cardiac connective tissue repair or tissue regeneration,
treating or ameliorating age-related or stress-related cardiomyopathy, and/or
treating or ameliorating a heart injury or dysfunction, an injury subsequent to (or following, or optionally from 1 minute to 12 hours after, or immediately after) a myocardial infarction (MI).

The details of one or more embodiments as provided herein are set forth in the accompanying drawings and the description below. Other features, objects, and advantages of embodiments as provided herein will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The drawings set forth herein are illustrative of embodiments as provided herein and are not meant to limit the scope of the invention as encompassed by the claims.

FIG. 1A-E illustrate images of Lin28A expressed in human and rodent cardiac tissue, and show related data:

FIG. 1A illustrates an image of adult human (normal 58 year old female) cardiac tissue demonstrating Lin28A is endogenous to both cardiomyocytes and cardiac interstitial cells, as shown by immunohistochemistry stain;

FIG. 1B illustrates an image of a protein separation gel showing that human protein quantity of Lin28A is significantly increased in aged normal heart tissue compared to neonatal cardiac tissue;

FIG. 1C graphically illustrates data showing quantification of Lin28A, normalized to GAPDH, which verifies aged human cardiac tissue has an increased amount of Lin28A protein compared to neonatal human cardiac tissue;

FIG. 1D illustrates an image of an adult FVB mouse cardiac tissue demonstrating that Lin28A is endogenous to cardiac interstitial cells, shown by immunohistochemistry stain; and

FIG. 1E graphically illustrates data showing surface Lin28A is equally present in freshly isolated male and female FVB adult isolate cardiac interstitial cells (CICs);

as discussed in further detail below.

FIG. 2A-H illustrate that oxidative stress promotes Lin28A^{S+} surface expression together with cardiac interstitial cells (CICs) senescence phenotype:

FIG. 2A illustrates data from an AMNIS IMAGESTREAM™ (Cytek Biosciences) flow cytometry, which was used to separate and count FVB total CICs for b-gal (Ch02—green), Brightfield (Ch04—grey), Lin28A^{S} (Ch05—red), side scatter (Ch06—pink), organized as Lin28A^{S+}/b-gal⁺ (top left), b-gal⁺ (top right) Lin28A^{S-} (bottom left), Lin28A^{S-}/b-gal⁻ (bottom right);

FIG. 2B graphically illustrates data of surface Lin28A or Bgal positive of total CICs, showing fresh isolate CICs are mostly Lin28A^{S-}/b-gal⁻, compared to 1% and 21% O₂;

FIG. 2C graphically illustrates data showing frequency of Lin28A^{S+} CICs is significantly increased in higher O₂ culture;

FIG. 2D graphically illustrates data showing that the size of Lin28A^{S+} CICs is significantly larger compared to Lin28A^{S-} CICs, regardless of O₂ culture;

FIG. 2E graphically illustrates data showing that the frequency of b-gal⁺ CICs is significantly increased in higher O₂ culture;

FIG. 2F graphically illustrates data showing that the size of b-gal⁺ CICs is significantly larger compared to b-gal⁻ CICs, regardless of O₂ culture;

FIG. 2G graphically illustrates data showing that Lin28A^{S+} CICs, b-gal⁺ is more likely present in 21% O₂ culture; and

FIG. 2H graphically illustrates data showing that Lin28A^{S+}/b-gal⁺ CICs are significantly larger in size, regardless of O₂ culture;

as discussed in further detail below.

FIG. 3A-M illustrate data showing that diploid content favored by low oxidative stress conditions tracks with CIC phenotypes of Lin28A^{S-} and small size:

FIG. 3A illustrates an image of data from an AMNIS IMAGESTREAM™ (Cytek Biosciences) flow cytometry, which was used to separate and count FVB Total CICs for Dapi (Ch01—purple), Brightfield (Ch04—grey), Lin28A^{S} (Ch05—red), side scatter (Ch06—pink), organized as mononuclear diploid (top left), binucleated diploid (top right) mononuclear tetraploid (bottom left), binucleated tetraploid (bottom right);

FIG. 3B graphically illustrates data showing that the frequency of Lin28A^{S+} CICs is significantly increased in higher O₂ culture;

FIG. 3C graphically illustrates data showing that Lin28A^{S+} of Total CIC expression is correlated with increased polyploidization;

FIG. 3D graphically illustrates data showing that Lin28A^{S+} CICs is correlated with increased polyploidization;

FIG. 3E graphically illustrates data showing that Lin28A^{S-} is correlated with diploid CICs;

FIG. 3F graphically illustrates data showing that the Total CIC population is significantly correlated with increased polyploidization in higher O₂ culture;

FIG. 3G graphically illustrates data showing that diploid CICs are consistently small in cell size, regardless of Lin28A^{S} or O₂ culture;

FIG. 3H graphically illustrates data showing that tetraploid CICs are of increased size, particularly when Lin28A^{S+}, regardless of O₂ culture;

FIG. 3I graphically illustrates data showing that tetraploid CICs are of increased size, particularly when Lin28A^{S+}, regardless of O₂ culture;

FIG. 3J illustrates an image showing that total CICs demonstrate binucleation and Lin28A^{S+}, as shown by immunohistochemistry;

FIG. 3K illustrates an image showing a zoom-in region of FIG. 3J, demonstrating binucleation and Lin28A^{S+};

FIG. 3L graphically illustrates data showing binucleation increases in Lin28A^{S+}, compared to Lin28A^{S-} CICs, in 1% O₂; and

FIG. 3M graphically illustrates data showing binucleation increases in Lin28A^{S+}, compared to Lin28A^{S-} CICs, in 21% O₂,

as discussed in further detail below.

FIG. 4A-C illustrate data showing that polyploidization of CICs occurs in response to chronic oxidative stress:

FIG. 4A graphically illustrates data showing that diploid CICs are maintained over passaging when exposed to 1% O₂ culture;

FIG. 4B graphically illustrates data showing that diploid CIC frequency is decreased, with increased tetraploid frequency over passaging when exposed to 21% O₂ culture; and

FIG. 4C graphically illustrates data showing that reactive oxygen species (ROS) is increased by passage 2 in CICs maintained in 21%, compared to 1%, O₂ culture;

as discussed in further detail below.

FIG. 5A-D illustrate data showing that surface Lin28A CICs are predominantly endothelial and hematopoietic lineage:

FIG. 5A-B illustrate a UMAP projection of Cardiac Interstitial Lin28^{S+/-} color-coded according to (FIG. 5A) unsupervised clustering of gene signatures and (FIG. 5B) as derived from either surface presence of Lin28;

FIG. 5C graphically illustrates data showing that cell contributions of Lin28^{S+/-} normalized to input of each main cell type as shown in UMAP (FIG. 5A-B); and

FIG. 5D illustrates a heatmap representing the differential expressed genes from Lin28^{S+/-} populations,

as discussed in further detail below.

FIG. 6A-F illustrate data showing that surface Lin28A CICs upregulate gene markers demonstrating a stress response:

FIG. 6A illustrates Gene ontology (GO) terms results from Gene Ontology analysis annotated by Biological Process, where the circle diameter represents the gene ratio from the 551/742 DEGs being expressed in the Lin28^{S+/-} cells, while significance level is color-coded according to heatmap scale;

FIG. 6B graphically illustrates data showing regulation of DNA-templated transcription in response to stress;

FIG. 6C graphically illustrates data showing regulation of stress-activated MAPK cascade;

FIG. 6D graphically illustrates data showing regulation of transcription from RNA polymerase II promoter in response to stress;

FIG. 6E graphically illustrates data showing response to endoplasmic reticulum stress; and

FIG. 6F graphically illustrates data showing a cellular response to oxidative stress in both Lin28^{S+/-} populations, where circle diameter represents the percentage of cells expressing a particular gene, while normalized average expression is represented by color intensity,

as discussed in further detail below.

FIG. 7A-F illustrate data showing that oxidative stress-induced phenotypic changes in CICs inhibited by anti-oxidant treatment

FIG. 7A graphically illustrates data showing the frequency of Lin28A^{S+} expression in Total CICs cultured in 21% O₂ is significantly decreased when treated with Trolox;

FIG. 7B graphically illustrates data showing the frequency of Bgal positive expression in Total CICs cultured in 21% O₂ is significantly decreased when treated with Trolox;

FIG. 7C graphically illustrates data showing the frequency of Lin28^{S+}/b-gal⁺ expression in Total CICs cultured in 21% O₂ is significantly decreased when treated with Trolox;

FIG. 7D graphically illustrates data showing the size of Lin28^{S+} CICs is reduced by Trolox treatment in a dose dependent manner;

FIG. 7E graphically illustrates data showing the size of Lin28^{S+}/b-gal⁺ CICs is reduced by Trolox treatment in a dose dependent manner; and

FIG. 7F graphically illustrates data showing that proliferation rate is higher in Total CICs treated with Trolox in a dose dependent manner,

as discussed in further detail below.

FIG. 8A-F illustrate data showing that Trolox increases diploid population and antagonizes conversion to higher ploidy:

FIG. 8A graphically illustrates data showing the frequency of diploid total CICs cultured in 21% O₂ is significantly increased when treated with Trolox in a dose dependent manner;

FIG. 8B graphically illustrates data showing the frequency of Lin28^{S+} CICs and polyploidization in total CICs cultured in 21% O₂ is significantly decreased when treated with Trolox in a dose dependent manner;

FIG. 8C graphically illustrates data showing the ploidy of Lin28^{S+} CICs cultured in 21% O₂ is unchanged with Trolox;

FIG. 8D graphically illustrates data showing the size of Lin28^{S+} CICs treated with Trolox in a dose dependent manner are significantly larger based on ploidy content;

FIG. 8E graphically illustrates data showing the frequency of diploid Lin28^{S-} CICs cultured in 21% O₂ is significantly increased when treated with Trolox in a dose dependent manner; and

FIG. 8E graphically illustrates data showing the size of Lin28^{S-} CICs treated with Trolox in a dose dependent manner are relatively unchanged based on ploidy content,

as discussed in further detail below.

FIG. 9A-B illustrate data showing that gating strategy on AMNIS™ for Lin28A^{S+/-} and b-galactosidase;

FIG. 9A illustrates AMNIS IMAGESTREAM™ (Cytek Biosciences) flow cytometry gating sorted based on size, in focus, b-gal, Lin28, and measured for frequency and area; and

FIG. 9B illustrates AMNIS IMAGESTREAM™ flow cytometry was used to separate and count FVB Total CICs for b-gal (Ch02—green), Brightfield (Ch04—grey), Lin28^{S+/-} (Ch05—red), side scatter (Ch06—pink), organized as Lin28A^{S+}/b-gal⁺ (top), b-gal⁺ (second to top) Lin28A^{S-} (third to top), Lin28A^{S-}/b-gal⁻ (bottom) as discussed in further detail below.

FIG. 10 graphically illustrates data showing that hydrogen peroxide increases frequency of Lin28A^{S+} CICs;

FIG. 11A-F illustrate data showing that chronic oxidative stress increases markers of senescence:

FIG. 11A graphically illustrates data showing that Lin28A^{S-} CICs, b-gal⁺ is more likely present in 21% O₂ culture;

FIG. 11B graphically illustrates data showing that Lin28A^{S-} CICs, b-gal⁺ CICs are significantly larger in size, regardless of O₂ culture;

FIG. 11C graphically illustrates data showing that b-gal⁺ CICs, Lin28A^{S+} is equally present in 1% and 21% O₂ culture;

FIG. 11D graphically illustrates data showing that b-gal⁺ CICs, Lin28A^{S+} CICs are significantly larger in size, regardless of O₂ culture;

FIG. 11E graphically illustrates data showing that b-gal⁻ CICs, nearly all cells are Lin28A^{S-} in fresh isolate, 1% and 21% O₂ culture; and

FIG. 11F graphically illustrates data showing that b-gal⁻ CICs, Lin28A^{S+} and Lin28A^{S-} CICs are approximately the same size, regardless of O₂ culture,

as discussed in further detail below.

FIG. 12A-B illustrates the gating strategy on AMNIS™ for Lin28A^{S+/-} and ploidy:

FIG. 12A illustrates AMNIS IMAGESTREAM™ (Cytek Biosciences) flow cytometry gating sorted based on size, in focus, Dapi, Lin28, ploidy and measured for frequency and area; and

FIG. 12B illustrates AMNIS IMAGESTREAM™ flow cytometry was used to separate and count FVB Total CICs for Dapi (Ch01—purple), Brightfield (Ch04—grey), Lin28A^{S+/-} (Ch05—red), side scatter (Ch06—pink), organized as mononuclear diploid (top left set), mononuclear tetraploid (bottom left set) binuclear diploid (top right set), binuclear tetraploid (bottom right set),

as discussed in further detail below.

FIG. 13A-B illustrate data showing that chronic oxidative stress increases polyploidization in both Lin28A^{S+/-}.

FIG. 13A graphically illustrates data showing that the frequency of diploid Lin28A^{S+} CICs is greater in 1% O₂ compared to 21% O₂; and

FIG. 13B graphically illustrates data showing that the frequency of diploid Lin28A^{S-} CICs is greater in 1% O₂ compared to 21% O₂,

FIG. 14A-D illustrates quality control for single cell RNA-SEQ experiment:

FIG. 14A-C illustrate a CELLRANGER 3.0.1™ quality control summary for Lin28S−/cKit− (FIG. 14A), Lin28S+/cKit+ (FIG. 14B), and Lin28S+/cKit− (FIG. 14A);

FIG. 14D illustrates a CELLRANGER 3.0.1™ library aggregation summary, as discussed in further detail below.

FIG. 15A-C illustrate data showing that unsupervised scRNAseq clustering reveals 14 clusters:

FIG. 15A illustrates an image of a principal component analysis (PCA);

FIG. 15B illustrates an image of a t-Distributed Stochastic Neighbor Embedding (t-SNE); and,

FIG. 15C illustrates an image of a Uniform Manifold Approximation and Projection (UMAP) dimensionality reduction projections of single cell data color coded by detected unsupervised clusters,

as discussed in further detail below.

FIG. 16 illustrates unsupervised scRNAseq clusters validated via expression of house-keeping genes, with the violin plots indicating expression of Gapdh, Actb, Rplp0, B2m and Ywhaz.

FIG. 17A-D illustrate data showing the cell type annotation of unsupervised clusters via expression of canonical cell markers, and violin plots identifying: FIG. 17A Endothelial, FIG. 17B Fibroblast, FIG. 17C Hematopoietic and FIG. 17D Myocyte cells based on expression of cell markers, as discussed in further detail below.

FIG. 18 illustrates cell type annotation of unsupervised clusters 6, 8 and 9 via cross-reference to the Tabula muris cell atlas. Heatmap representing cell type correlation scores between the cluster expression matrix with the Tabula muris cell expression data, as discussed in further detail below.

FIG. 19 graphically illustrates cell contributions of Lin28^{S+/-} normalized to input of each main cell type and cluster as shown in UMAP, (see also FIG. 1A, 1B), as discussed in further detail below.

FIG. 20 illustrates supplement Table 1, showing polyploidization in CICs occurs in response to chronic oxidative stress, as discussed in further detail below.

FIG. 21 illustrates supplement Table 2, showing data that Trolox inhibits oxidative stress-induced phenotype changes in CICs, as discussed in further detail below.

FIG. 22 illustrates supplement Table 3, showing that Trolox increases diploid population and antagonizes conversion to higher ploidy, as discussed in further detail below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments as provided herein, and should not be interpreted as a limitation on the scope of the invention.

### DETAILED DESCRIPTION

In alternative embodiments, provided are methods for: removing cardiac cells impaired or functionally diminished by stress or age, wherein optionally cardiac cells impaired or functionally diminished have reduced proliferative capacity and/or polyploidy, initiating, inducing or accelerating a cardiac muscle repair or tissue regeneration, a cardiac vasculature repair or tissue regeneration or a cardiac connective tissue repair or tissue regeneration, treating or ameliorating age-related or stress-related cardiomyopathy, and/or treating or ameliorating a heart injury, an injury subsequent to (or following, or optionally from 1 minute to 12 hours after, or immediately after) a myocardial infarction (MI), comprising: administering to an individual in need thereof a composition or treatment that ablates or inactivates Lin28+ cardiac cells, including Lin28+ cardiac stem cells or cardiac progenitor cells, or administrating to a patient a product of manufacture as provided herein.

While the invention is not limited by any particular mechanism of action, because Lin28A redistribution is indicative of stress response in cardiac interstitial cells (CICs) associated with aging and senescence, removal or ablation of Lin28A+ cells in the heart has a therapeutic effect, slowing if not actually reversing aging and senescence associated with CIC Lin28A+ expression.

As discussed in Example 1, localization of Lin28A was assessed by multiple experimental analyses and treatment conditions and correlated to oxidative stress, senescence, and ploidy in adult murine CICs. Surface Lin28A expression is present on 5% of fresh CICs and maintained through passage 2, increasing to 21% in hyperoxic conditions but lowered to 14% in physiologic normoxia. Surface Lin28A is coincident with elevated b-galactosidase (b-gal) expression in CICs expanded in hyperoxia, and also increases with polyploidization and binucleation of CICs regardless of oxygen culture. Transcriptional profiling of CICs using single cell RNASeq reveals upregulation of pathways associated with oxidative stress in CICs exhibiting surface Lin28A. Induction of surface Lin28A by oxidative stress is blunted by treatment of cells with the antioxidant Trolox (6-hydroxy-2,5,7,8-tetramethylchroman-2-carboxylic acid; a chromanol that is 6-hydroxychromane which is substituted by a carboxy group at position 2 and by methyl groups at positions 2, 5, 7, and 8, and is a cell-permeable, water-soluble analogue of vitamin E, it is used as a standard for measuring the antioxidant capacity of complex mixtures) in a dose-dependent manner, with 300 uM Trolox exposure maintaining characteristics of freshly isolated CICs possessing low expression of surface Lin28A and b-gal with predominantly diploid content. Based on this data it can be concluded that surface Lin28A is a marker of environmental oxidative stress in CICs and antioxidant treatment as provided herein antagonizes this phenotype. The biological significance of Lin28 surface expression and consequences for myocardial responses provides important insights regarding mitigation of cardiac stress and aging.

### Polypeptides and Antibodies

In alternative embodiments, provided are compositions and methods for inhibiting or decreasing the expression or activity of an Lin28 polypeptide in a tissue or an organ, or ablating or removing Lin28-expressing cells from the tissue or organ, optionally in an organ-specific or an organ-selective manner, for example, targeting the heart, by, for example administering the peptide or a polypeptide, for example, an antibody or fragment thereof or equivalent thereof, capable of specifically binding or otherwise inhibiting the activity or expression of a Lin28, for example, human Lin28.

In alternative embodiments, the administered peptides or polypeptides, for example, anti-Lin28 antibodies or fragments thereof, are linked with or conjugated to a cytotoxic payload or agent such for example a cytotoxic compound or cytotoxic radionuclide, wherein optionally the cytotoxic compound comprises calicheamicin, duocarymycin or a ribosome-inactivating protein (RIP), optionally ricin.

In alternative embodiments, the administered peptides or polypeptides, for example, anti-Lin28 antibodies or fragments thereof, are linked with or conjugated to a radionuclide such as astatine-211, yttrium-90, lutetium-177 or iodine-131.

In alternative embodiments, the cytotoxic agent or payload is covalently linked to the anti-Lin28+ antibody, for example, by a linker such as disuccinimidyl suberate (DSS), or the cytotoxic payload is conjugated to the anti-Lin28+ antibody using a metal chelators, optionally diethylenetriaminepentaacetic acid (DTPA) or 1,4,7,10-tetraazacyclododecane-1,4,7,10-tetraacetic acid (DOTA), or equivalents, and the like.

Antibodies or fragments thereof capable of specifically binding Lin28 can be designed using Homo sapiens Lin28 proteins or fragments thereof, for example, having an amino acid sequence:

(SEQ ID NO: 1)
MGSVSNQQFAGGCAKAAEEAPEEAPEDAARAADEPQLLHGAGICKWFNV
RMGFGFLSMTARAGVALDPPVDVFVHQSKLHMEGFRSLKEGEAVEFTFK
KSAKGLESIRVTGPGGVFCIGSERRPKGKSMQKRRSKGDRCYNCGGLDH
HAKECKLPPQPKKCHFCQSISHMVASCPLKAQQGPSAQGKPTYFREEEE
EIHSPTLLPEAQN

(see Strausberg, et al (2002) Proc. Natl. Acad. Sci. U.S.A. 99 (26), 16899-16903).

### Inhibitory or Antisense Nucleic Acid Molecules

In alternative embodiments, provide are methods for removing cardiac cells impaired or functionally diminished by stress or age, initiating, inducing or accelerating a cardiac muscle repair or tissue regeneration, a cardiac vasculature repair or tissue regeneration or a cardiac connective tissue repair or tissue regeneration, comprising administering a nucleic acid composition that is capable of inhibiting or decreasing the activity of Lin28+, or ablating or killing a Lin28+-expressing cardiac cell, for example, an antisense nucleic acid such as an antisense morpholino oligonucleotide (MO), or an miRNA, an siRNA and the like.

In alternative embodiments, compositions and methods as provided herein comprise use of an inhibitory nucleic acid molecule or an antisense oligonucleotide inhibitory to expression of a Lin28 polypeptide. In alternative embodiments, compositions and methods as provided herein comprise use of an inhibitory nucleic acid molecule or antisense oligonucleotide inhibitory to expression of an Lin28, comprising: an RNAi inhibitory nucleic acid molecule, a double-stranded RNA (dsRNA) molecule, a small interfering RNA (siRNA), a microRNA (miRNA) and/or a short hairpin RNA (shRNA), or a ribozyme.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol. Appl. Pharmacol. 144:189-197; Antisense Therapeutics, ed. Agrawal (Humana Press, Totowa, N.J., 1996). Antisense oligonucleotides having synthetic DNA backbone analogues can also include phosphoro-dithioate, methylphosphonate, phosphoramidate, alkyl phosphotriester, sulfamate, 3′-thioacetal, methylene (methylimino), 3′-N-carbamate, and morpholino carbamate nucleic acids.

Antisense nucleic acid sequences can be designed using wild type sequences, for example, using the human Lin28 coding sequence:

| SEQ ID NO: 2 | |
|---|---|
| 1 | cctttgcctt cggacttctc cggggccagc agccgcccga ccaggggccc ggggccacgg |
| 61 | gctcagccga cgaccatggg ctccgtgtcc aaccagcagt ttgcaggtgg ctgcgccaag |
| 121 | gcggcagaag aggcgcccga ggaggcgccg gaggacgcgg cccgggcggc ggacgagcct |
| 181 | cagctgctgc acggtgcggg catctgtaag tggttcaacg tgcgcatggg gttcggcttc |
| 241 | ctgtccatga ccgcccgcgc cggggtcgcg ctcgaccccc cagtggatgt ctttgtgcac |
| 301 | cagagtaagc tgcacatgga agggttccgg agcttgaagg agggtgaggc agtggagttc |
| 361 | acctttaaga agtcagccaa gggtctggaa tccatccgtg tcaccggacc tggtggagta |
| 421 | ttctgtattg ggagtgagag gcggccaaaa ggaaagagca tgcagaagcg cagatcaaaa |
| 481 | ggagacaggt gctacaactg tggaggtcta gatcatcatg ccaaggaatg caagctgcca |
| 541 | ccccagccca agaagtgcca cttctgccag agcatcagcc atatggtagc ctcatgtccg |
| 601 | ctgaaggccc agcagggccc tagtgcacag ggaaagccaa cctactttcg agaggaagaa |
| 661 | gaagaaatcc acagccctac cctgctcccg gaggcacaga attgagccac aatgggtggg |
| 721 | ggctattctt ttgctatcag gaagttttga ggagcaggca gagtggagaa agtgggaata |
| 781 | gggtgcattg gggctagttg gcactgccat gtatctcagg cttgggttca caccatcacc |
| 841 | ctttcttccc tctaggtggg gggaaagggt gagtcaaagg aactccaacc atgctctgtc |
| 901 | caaatgcaag tgagggttct gggggcaacc aggagggggg aatcacccta caacctgcat |
| 961 | actttgagtc tccatcccca gaatttccag cttttgaaag tggcctggat agggaagttg |
| 1021 | ttttcctttt aaagaaggat atataataat tcccatgcca gagtgaaatg attaagtata |
| 1081 | agaccagatt catggagcca agccactaca ttctgtggaa ggagatctct caggagtaag |
| 1141 | cattgttttt ttttcacatc ttgtatcctc atacccactt ttgggatagg gtgctggcag |
| 1201 | ctgtcccaag caatgggtaa tgatgatggc aaaaagggtg tttgggggaa cagctgcaga |
| 1261 | cctgctgctc tatgctcacc cccgccccat tctgggccaa tgtgatttta tttatttgct |
| 1321 | cccttggata ctgcaccttg ggtcccactt tctccaggat gccaactgca ctagctgtgt |
| 1381 | gcgaatgacg tatcttgtgc attttaactt tttttcctta atataaatat tctggttttg |
| 1441 | tatttttgta tattttaatc taaggccctc atttcctgca ctgtgttctc aggtacatga |
| 1501 | gcaatctcag ggatagccag cagcagctcc aggtctgcgc agcaggaatt actttttgtt |
| 1561 | gtttttgcca ccgtggagag caactatttg gagtgcacag cctattgaac tacctcattt |
| 1621 | ttgccaataa gagctggctt ttctgccata gtgtcctctt gaaaccccct ctgccttgaa |
| 1681 | aatgttttat gggagactag gttttaactg ggtggcccca tgacttgatt gccttctact |
| 1741 | ggaagattgg gaattagtct aaacaggaaa tggtggtaca cagaggctag gagaggctgg |
| 1801 | gcccggtgaa aaggccagag agcaagccaa gattaggtga gggttgtcta atcctatggc |
| 1861 | acaggacgtg ctttacatct ccagatctgt tcttcaccag attaggttag gcctaccatg |
| 1921 | tgccacaggg tgtgtgtgtg tttgtaaaac tagagttgct aaggataagt ttaaagacca |
| 1981 | atacccctgt acttaatcct gtgctgtcga gggatggata tatgaagtaa ggtgagatcc |
| 2041 | ttaacctttc aaaattttcg ggttccaggg agacacacaa gcgagggttt tgtggtgcct |
| 2101 | ggagcctgtg tcctgccctg ctacagtagt gattaatagt gtcatggtag ctaaaggaga |
| 2161 | aaaagggggt ttcgtttaca cgctgtgaga tcaccgcaaa cctaccttac tgtgttgaaa |
| 2221 | cgggacaaat gcaatagaac gcattgggtg gtgtgtgtct gatcctgggt tcttgtctcc |
| 2281 | cctaaatgct gccccccaag ttactgtatt tgtctgggct ttgtaggact tcactacgtt |
| 2341 | gattgctagg tggcctagtt tgtgtaaata taatgtattg gtctttctcc gtgttctttg |
| 2401 | ggggttttgt ttacaaactt ctttttgtat tgagagaaaa atagccaaag catctttgac |
| 2461 | agaaggttct gcaccaggca aaaagatctg aaacattagt ttggggggcc ctcttcttaa |
| 2521 | agtggggatc ttgaaccatc ctttcttttg tattcccctt cccctattac ctattagacc |
| 2581 | agatcttctg tcctaaaaac ttgtcttcta ccctgccctc ttttctgttc acccccaaaa |
| 2641 | gaaaacttac acacccacac acatacacat ttcatgcttg gagtgtctcc acaactctta |
| 2701 | aatgatgtat gcaaaaatac tgaagctagg aaaaccctcc atcccttgtt ccaacctcc |
| 2761 | taagtcaaga ccattaccat ttctttcttt cttttttttt tttttttaaa atggagtctc |
| 2821 | actgtgtcac ccaggctgga gtgcagtggc atgatcggct cactgcagcc tctgcctctt |
| 2881 | gggttcaagt gattctcctg cctcagcctc ctgagtagct gggatttcag gcacccgcca |
| 2941 | cactcagcta atttttgtat ttttagtaga gacggggttt caccatgttg tccaggctgg |
| 3001 | tctggaactc ctgacctcag gtgatctgcc caccttggct tcccaaagtg ctgggattac |
| 3061 | aggcatgagc caccatgctg ggccaaccat ttcttggtgt attcatgcca aacacttaag |
| 3121 | acactgctgt agcccaggcg cggtggctca cacctgtaat cccagcactt tggaaggctg |
| 3181 | aggcgggcgg atcacaaggt cacgagttca aaactatcct ggccaacaca gtgaaacccc |
| 3241 | gtctctacta aaatacaaaa aaattagccg ggtgtggtgg tgcatgcctt tagtcctagc |
| 3301 | tattcaggag gctgaggcag gggaatcgct tgaacccgag aggcagaggt tgcagtgagc |
| 3361 | tgagatcgca ccactgcact ccagcctggt tacagagcaa gactctgtct caaacaaaac |
| 3421 | aaaacaaaac aaaaacacac tactgtattt tggatggatc aaacctcctt aattttaatt |
| 3481 | tctaatccta aagtaaagag atgcaattgg gggccttcca tgtagaaagt ggggtcagga |
| 3541 | ggccaagaaa gggaatatga atgtatatcc aagtcactca ggaactttta tgcaggtgct |
| 3601 | agaaacttta tgtcaaagtg gccacaagat tgtttaatag gagacgaacg aatgtaactc |
| 3661 | catgtttact gctaaaaacc aaagctttgt gtaaaatctt gaatttatgg ggcgggaggg |
| 3721 | taggaaagcc tgtacctgtc tgtttttttc ctgatccttt tccctcattc ctgaactgca |
| 3781 | ggagactgag cccctttggg ctttggtgac cccatcactg gggtgtgttt atttgatggt |
| 3841 | tgattttgct gtactgggta cttcctttcc cattttctaa tcatttttta acacaagctg |
| 3901 | actcttccct tcccttctcc tttccctggg aaaatacaat gaataaataa agacttattg |
| 3961 | gtacgcaaac tgtca |

### RNA Interference (RNAi)

In alternative embodiments, provided are RNAi inhibitory nucleic acid molecules capable of decreasing or inhibiting expression of one or a set of Lin28 transcripts or proteins, optionally in a heart-specific or heart-selective manner, and including for example decreasing or inhibiting expression of the transcript (mRNA, message) or isoform or isoforms thereof. In one aspect, the RNAi molecule comprises a double-stranded RNA (dsRNA) molecule. The RNAi molecule can comprise a double-stranded RNA (dsRNA) molecule, for example, siRNA, miRNA (microRNA) and/or short hairpin RNA (shRNA) molecules.

In alternative aspects, the RNAi is about 11, 12, 13, 14, 15, 16, 17, 18, 19, 20, 21, 22, 23, 24, 25 or more duplex nucleotides in length. While the methods provided herein are not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, for example, siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence.

In one aspect, intracellular introduction of the RNAi (for example, miRNA or siRNA) is by internalization of a target cell specific ligand bonded to an RNA binding protein comprising an RNAi (for example, microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, for example, U.S. Patent App. Pub. Nos. 20060030003; 20060025361; 20060019286; 20060019258. In one aspect, provided are lipid-based formulations for delivering, for example, introducing nucleic acids used in methods as provided herein, as nucleic acid-lipid particles comprising an RNAi molecule to a cell, see, for example, U.S. Patent App. Pub. No. 20060008910.

Methods for making and using RNAi molecules, for example, siRNA and/or miRNA, for selectively degrade RNA are well known in the art, see, for example, U.S. Pat. Nos. 6,506,559; 6,511,824; 6,515,109; 6,489,127.

Methods for making expression constructs, for example, vectors or plasmids, from which an inhibitory polynucleotide (for example, a duplex siRNA) is transcribed are well known and routine. A regulatory region (for example, promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, for example, a Lin28 coding sequence or transcriptional activation sequence, is inserted between two promoters (for example, mammalian, viral, human, tissue specific, constitutive or other type of promoter) such that transcription occurs bidirectionally and will result in complementary RNA strands that may subsequently anneal to form an inhibitory siRNA used to practice methods as provided herein.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice methods as provided herein.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, for example, of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

### Inhibitory Ribozymes

In alternative embodiment, compositions and methods as provided herein comprise use of ribozymes capable of binding and inhibiting, for example, decreasing or inhibiting, expression of one or a set of Lin28 transcripts or proteins, or isoforms or isoforms thereof, optionally in a heart-specific or heart-selective manner.

These ribozymes can inhibit a gene's activity by, for example, targeting a genomic DNA or an mRNA (a message, a transcript). Strategies for designing ribozymes and selecting a gene-specific antisense sequence for targeting are well described in the scientific and patent literature, and the skilled artisan can design such ribozymes using these reagents. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

Materials or delivery adjuvants can be used to enhance cell retention and their longevity once delivered to a heart, for example, by administration with or formulated with (for example, mixed with) a gel or a hydrogel, such as a chitosan-based hydrogel, for example, as described in Kurdi et al. Congest Heart Fail. 2010 May-June; 16(3):132-5, or any biocompatible scaffold, for example, as described in U.S. Pat. Nos. 8,871,237; 8,753,391; 8,802,081; 8,691,543, or Pagliari et al. Curr Med Chem. 2013; 20(28):3429-47, or biomimetic support, for example, as described in Karam et al. Biomaterials. 2012 August; 33(23):5683-95.

### In Vivo Delivery of Anti Lin28 Nucleic Acids

In alternative embodiments, provided are compositions and methods for delivering nucleic acids encoding anti-Lin28 peptides or polypeptides, or anti-Lin28 nucleic acids, or vectors or recombinant viruses having contained therein these nucleic acids. In alternative embodiments, the nucleic acids, vectors or recombinant viruses are designed for in vivo or CNS delivery and expression.

In alternative embodiments, provided are compositions and methods for the delivery and controlled expression of an anti-Lin28 nucleic acid or gene, or an expression vehicle (for example, vector, recombinant virus, and the like) comprising (having contained therein) an anti-Lin28 nucleic acid or gene.

In alternative embodiments, the provided are methods for being able to turn on and turn off Lin28-expressing nucleic acid or gene expression easily and efficiently for tailored treatments and insurance of optimal safety.

In alternative embodiments, provided are expression vehicles, vectors, recombinant viruses and the like for in vivo expression of an anti-Lin28 nucleic acid or gene to practice the methods as provide herein. In alternative embodiments, the anti-Lin28 nucleic acids (such as RNA or DNA), expression vehicles, vectors, recombinant viruses and the like expressing an anti-Lin28 nucleic acid or gene can be delivered by intravitreal injection or intramuscular (IM) injection (using for example, RNA in liposomes), by intravenous (IV) injection, by subcutaneous injection, by inhalation, by a biolistic particle delivery system (for example, a so-called “gene gun”), and the like, for example, as an outpatient, for example, during an office visit.

In alternative embodiments, this “peripheral” mode of delivery, for example, expression vehicles, vectors, recombinant viruses and the like injected intravitreal, IM or IV, can circumvent problems encountered when genes or nucleic acids are expressed directly in an organ (for example, an eye, the brain or into the CNS) itself. Sustained secretion of an anti-Lin28 polypeptide or nucleic acid in the bloodstream or general circulation also circumvents the difficulties and expense of administering proteins by infusion.

In alternative embodiments a recombinant virus (for example, a long-term virus or viral vector), or a vector, or an expression vector, and the like, can be injected, for example, in a systemic vein (for example, IV), or by intravitreal, intramuscular (IM) injection, by inhalation, or by a biolistic particle delivery system (for example, a so-called “gene gun”), for example, as an outpatient, for example, in a physician's office. In alternative embodiments, days or weeks later (for example, four weeks later), the individual, patient or subject is administered (for example, inhales, is injected or swallows), a chemical or pharmaceutical that induces expression of anti-Lin28 nucleic acids or genes; for example, an oral antibiotic (for example, doxycycline or rapamycin) is administered once daily (or more or less often), which will activate the expression of the gene. In alternative embodiments, after the “activation”, or inducement of expression (for example, by an inducible promoter) of the nucleic acid or gene, an anti-Lin28 nucleic acid is synthesized and released into the subject's circulation (for example, into the blood), and subsequently has favorable physiological effects, for example, therapeutic or prophylactic, that benefit the individual or patient (for example, benefit heart function). When the physician or subject desires discontinuation of the anti-Lin28 treatment, the subject simply stops taking the activating chemical or pharmaceutical, for example, antibiotic.

Alternative embodiments comprise use of “expression cassettes” comprising or having contained therein a nucleotide sequence used to practice methods provided herein, for example, an anti-Lin28 nucleic acid, which can be capable of affecting expression of the nucleic acid, for example, as a structural gene or a transcript (for example, encoding Lin28 protein) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a retinal cell. Promoters used in the constructs provided herein include cis-acting transcriptional control elements and regulatory sequences that are involved in regulating or modulating the timing and/or rate of transcription of a nucleic acid, for example, an anti-Lin28 nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an anti-Lin28 nucleic acid, under the influence of environmental conditions, administered chemical agents, or developmental conditions.

### Gene Therapy and Gene Delivery Vehicles

In alternative embodiments, methods of the invention comprise use of nucleic acid (for example, an anti-Lin28 nucleic acid) delivery systems to deliver a payload of an anti-Lin28 nucleic acid, transcript or message, to a cell or cells in vitro, ex vivo, or in vivo, for example, as gene therapy delivery vehicles.

In alternative embodiments, expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV, or a neurotropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14(3):316-27. Epub 2006 Jul. 7; Choi, et al., Curr. Gene Ther. 2005 June; 5(3):299-310.

For example, in alternative embodiments, serotypes AAV-8, AAV-9, AAV-DJ or AAV-DJ/8™ (Cell Biolabs, Inc., San Diego, CA), which have increased uptake in brain tissue in vivo, are used to deliver an anti-Lin28 nucleic acid payload for expression in the CNS. In alternative embodiments, the following serotypes, or variants thereof, are used for targeting a specific tissue:

| Tissue | Optimal Serotype |
|---|---|
| CNS | AAV1, AAV2, AAV4, AAV5, AAV8, AAV9 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

In alternative embodiments, the rhesus-derived AAV AAVrh.10hCLN2 or equivalents thereof can be used, wherein the rhesus-derived AAV may not be inhibited by any pre-existing immunity in a human; see for example, Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh.10hCLN2 to the CNS of rats and non-human primates at doses scalable to humans has an acceptable safety profile and mediates significant payload expression in the CNS.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, methods provided herein can comprise screening of patient candidates for AAV-specific NAbs prior to treatment, such as with an AAV8 capsid component, to facilitate individualized treatment design and enhance therapeutic efficacy; see, for example, Sun, et al., J. Immunol. Methods. 2013 Jan. 31; 387(1-2):114-20, Epub 2012 Oct. 11.

In alternative embodiments, the anti-Lin28 nucleic acid as delivered in vivo using methods as provided herein can be in the form of, or comprise, an RNA, for example, mRNA, which can be formulated in a lipid formulation or a liposome and injected for example intramuscularly (IM), for example using formulations and methods as described in U.S. patent application no. US 20210046173 A1, which describes delivering to a subject (for example, via intramuscular administration) the anti-Lin28 nucleic acid that comprises a RNA (for example, mRNA) that comprises an open reading frame (ORF) that comprises (or consists of, or consists essentially of) or encodes for anti-Lin28 nucleic acid; wherein optionally the RNA (or the DNA-carrying expression vehicle) is formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Lin28 Inhibitory Compounds

In alternative embodiments, provided are compounds inhibitory to Lin28 function and/or expression, or pharmaceutical compositions and formulations comprising same, or for practicing methods as provided herein, for example, methods for enhancing or accelerating heart function, or inhibiting or decreasing the activity of Lin28+, or ablating or killing a Lin28+-expressing cardiac cell in vivo.

In alternative embodiments, a compound inhibitory to Lin28 function and/or expression is 6-hydroxy-DL-DOPA (see Lightfoot et al, Org. Biomol. Chem., 2016, 14, 10208-10216); or N-methyl-N-[3-(3-methyl[1,2,4]triazolo[4,3-b]pyridazin-6-yl)phenyl]acetamide (see Roos et al, ACS Chem Biol (2006) Vol 11(10):2773-2781); or, an enantiomer or stereoisomer thereof.

In alternative embodiments, a compound inhibitory to Lin28 function and/or expression is TPEN, LI20 or LI171 (see Wang et al, Cell Rep. 2018 Jun. 5; 23(10): 3091-3101):

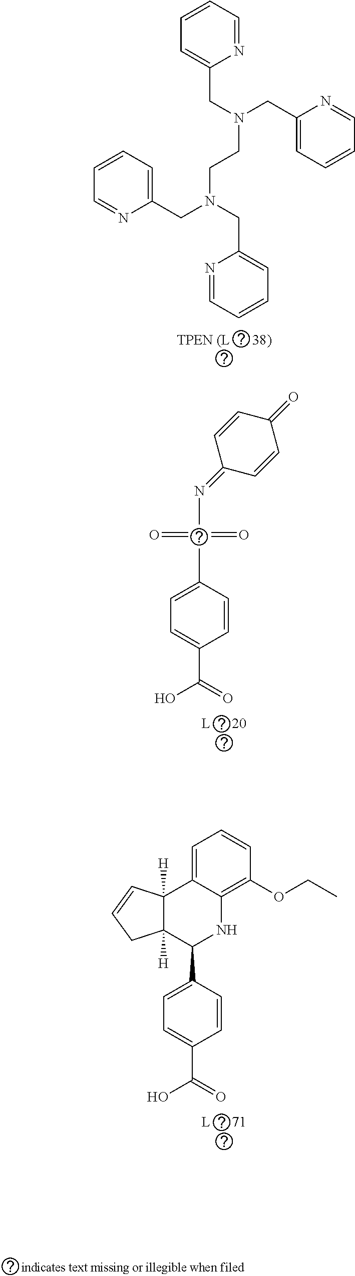

In alternative embodiments, a compound inhibitory to Lin28 function and/or expression is CL285032 (N-methyl-N-[3-(3-methyl[1,2,4]triazolo[4,3-b]-pyridazin-6-yl)phenyl]acetamide), or enantiomers or sterioisomers thereof, see for example Chen et al Cancer Immunol Res (2019) 7 (3): 487-497, describing the synthesis of CL285032:

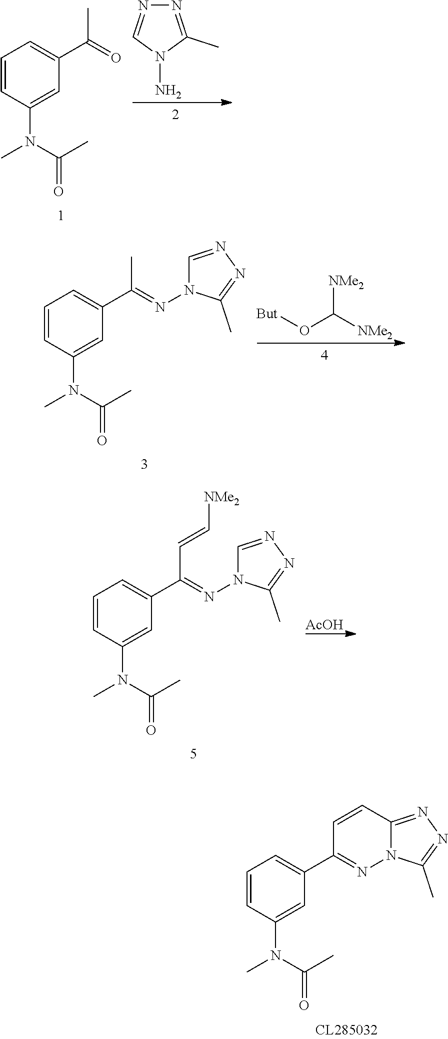

### Pharmaceutical Compositions and Formulations

In alternative embodiments, provided are compounds, nucleic acids and/or polypeptides such as antibodies inhibitory to Lin28, or pharmaceutical compositions and formulations comprising same, or for practicing methods as provided herein, for example, methods for enhancing or accelerating heart function, or inhibiting or decreasing the activity of Lin28+, or ablating or killing a Lin28+-expressing cardiac cell in vivo.

In alternative embodiments, compounds and compositions used to practice the methods as provided herein are formulated with a pharmaceutically acceptable carrier. In alternative embodiments, the pharmaceutical compositions used to practice the methods as provided herein can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co, Easton PA (“Remington's”).

Therapeutic agents used to practice the methods as provided herein can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods as provided herein include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods as provided herein can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, for example, sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, for example, gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods as provided herein can also be used orally using, for example, push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (for example, a composition used to practice the methods as provided herein) in admixture with excipients suitable for the manufacture of aqueous suspensions. Such excipients include a suspending agent, such as sodium carboxymethylcellulose, methylcellulose, hydroxypropyl-methylcellulose, sodium alginate, polyvinylpyrrolidone, gum tragacanth and gum acacia, and dispersing or wetting agents such as a naturally occurring phosphatide (for example, lecithin), a condensation product of an alkylene oxide with a fatty acid (for example, polyoxyethylene stearate), a condensation product of ethylene oxide with a long chain aliphatic alcohol (for example, heptadecaethylene oxycetanol), a condensation product of ethylene oxide with a partial ester derived from a fatty acid and a hexitol (for example, polyoxyethylene sorbitol mono-oleate), or a condensation product of ethylene oxide with a partial ester derived from fatty acid and a hexitol anhydride (for example, polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents used to practice the methods as provided herein. Oil-based suspensions can be formulated by suspending an active agent in a vegetable oil, such as arachis oil, olive oil, sesame oil or coconut oil, or in a mineral oil such as liquid paraffin; or a mixture of these. See for example, U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations as provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing methods provided herein, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing methods provided herein, the pharmaceutical compounds can be delivered by transdermally, by a topical route, formulated as applicator sticks, solutions, suspensions, emulsions, gels, creams, ointments, pastes, jellies, paints, powders, and aerosols.

In practicing methods provided herein, the pharmaceutical compounds can also be delivered as nanoparticles or microspheres for regulated, for example, fast or slow release in the body. For example, nanoparticles or microspheres can be administered via intradermal injection of drug which slowly release subcutaneously; see Rao (1995) J. Biomater Sci. Polym. Ed. 7:623-645; as biodegradable and injectable gel formulations, see, for example, Gao (1995) Pharm. Res. 12:857-863 (1995); or, as microspheres for oral administration, see, for example, Eyles (1997) J. Pharm. Pharmacol. 49:669-674. Nanoparticles can also be given intravenously, for example nanoparticles with linkage to biological molecules as address tags could be targeted to specific tissues or organs.

In practicing methods provided herein, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (for example, substantially uninterrupted introduction into a blood vessel for a specified period of time).

The pharmaceutical compounds and formulations used to practice the methods as provided herein can be lyophilized. Provided are a stable lyophilized formulation comprising a composition as provided herein, which can be made by lyophilizing a solution comprising a pharmaceutical as provided herein and a bulking agent, for example, mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, for example, U.S. patent app. no. 20040028670.

The compositions and formulations used to practice the methods as provided herein can be delivered by the use of liposomes or nanoliposomes. By using liposomes, particularly where the liposome surface carries ligands specific for target cells, for example, liver cells, or are otherwise preferentially directed to a specific organ or tissues, for example, liver, a heart, a kidney, muscle, bone, skin, trachea, arterial or venous blood vessels, intestine, spinal cord, nerve or a brain, one can focus the delivery of the active agent into target cells in vivo. See, for example, U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods as provided herein can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions as provided herein are administered in an amount sufficient to for for example, enhancing or accelerating liver regeneration, optionally enhancing or accelerating liver regeneration after tissue injury or liver resection; enhancing or accelerating tissue or organ repair, optionally enhancing or accelerating tissue or organ repair after a trauma, an injury or an infection, wherein optionally the injury is an ischemia-reperfusion injury, for example, a heart attack or a stroke; or, reducing the extent of or abolishing ischemia-reperfusion injury in a tissue or organ, for example, a normal liver or a fatty liver, in an individual, or in a cadaver or donor tissue or organ or transplant tissue or organ, for example, or a cadaver or donor heart, lung, kidney, skin, or pancreas intended for transplant, in need thereof.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods as provided herein can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ or tissue. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, for treating heart failure or heart attack. For example, the methods and/or compositions and formulations as provided herein can be co-formulated with and/or co-administered with antibiotics (for example, antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (for example, a ficolin), carbohydrate-binding domains, and the like and combinations thereof.

### Nanoparticles, Nanolipoparticles and Liposomes

Also provided are nanoparticles, nanolipoparticles, vesicles and liposomal membranes comprising compounds used to practice the methods as provided herein, for example, to deliver compositions used to practice methods as provided herein (for example, Lin28 inhibitors) to mammalian, for example, heart tissue, in vivo, in vitro or ex vivo. In alternative embodiments, these compositions are designed to target specific molecules, including biologic molecules, such as polypeptides, including cell surface polypeptides, for example, for targeting a desired cell type, for example, a liver cell, or a liver endothelial or sinusoidal cell, and the like.

Provided are multilayered liposomes comprising compounds used to practice methods as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, for example, as described in Park, et al., U.S. Pat. Pub. No. 20070042031, including method of producing a liposome by encapsulating an active agent (for example, Lin28-inhibiting nucleic acids and polypeptides), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, for example, for targeting delivery of a compound (for example, Lin28-inhibiting nucleic acid or polypeptide) used to practice methods as provided herein to a desired cell type (for example, a heart, as described for example, in U.S. Pat. Pub. No. 20070110798.

Provided are nanoparticles comprising compounds (for example, Lin28-inhibiting nucleic acids and polypeptides) used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent used to practice a method as provided herein or a fat-solubilized water-soluble active agent to act with a bivalent or trivalent metal salt.

In one embodiment, solid lipid suspensions can be used to formulate and to deliver compositions used to practice methods as provided herein to mammalian, for example, liver, cells in vivo, in vitro or ex vivo, as described, for example, in U.S. Pat. Pub. No. 20050136121.

### Delivery Vehicles

In alternative embodiments, any delivery vehicle can be used to practice the methods as provided herein, for example, to deliver compositions methods as provided herein (for example, Lin28 inhibitors) to mammalian, for example, human, liver cells in vivo, in vitro or ex vivo. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate a composition used to practice a method as provided herein, for example as described, for example, in U.S. Pat. Pub. No. 20040151766.

In one embodiment, a composition used to practice methods as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, for example, using any electroporation system as described for example in U.S. Pat. Nos. 7,109,034; 6,261,815; 5,874,268.

### Dosaging

The pharmaceutical compositions and formulations used to practice methods and uses as provided herein can be administered for prophylactic and/or therapeutic treatments, for example, to treat, ameliorate, protect against heart failure. In therapeutic applications, compositions are administered to a subject already suffering from a disease, condition, infection or defect in an amount sufficient to cure, alleviate or partially arrest the clinical manifestations of the disease, condition, infection or disease and its complications (a “therapeutically effective amount”), including for example, heart failure or cardiac senescence. For example, in alternative embodiments, anti-Lin28 nucleic acid- or polypeptide-comprising pharmaceutical compositions and formulations as provided herein are administered to an individual in need thereof in an amount sufficient to treat, ameliorate, protect against, diseases and conditions as described herein.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, viral vectors such as adenovirus or AAV vectors are administered to an individual in need therein, and in alternative embodiment the dosage administered to a human comprises: a dose of about 2×10¹² vector genomes per kg body weight (vg/kg), or between about 10¹⁰ and 10¹⁴ vector genomes per kg body weight (vg/kg), or about 10⁹, 10¹⁰, 10¹¹, 10¹², 10¹³, 10¹⁴, 10¹⁵, or more vg/kg, which can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the vectors are contained in a nanoparticle, a particle, a micelle or a liposome or lipoplex, a polymersome, a polyplex or a dendrimer. In alternative embodiments, these dosages are administered once a day, once a week, or any variation thereof as needed to decrease in vivo expression levels of Lin28 cardiac cells, which can be monitored by measuring actually expression of Lin28 or by monitoring of therapeutic effect, for example, to treat, ameliorate, protect against, reverse or decrease the severity or duration of a disease or condition as provided herein. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### Bioisosteres of Compounds

In alternative embodiment, also provided are bioisosteres of compounds and polypeptides used to practice the methods provided herein, for example, polypeptides having a Lin28 inhibitory activity. Bioisosteres used to practice methods as provided herein include bioisosteres of, for example, anti-Lin28 nucleic acids and polypeptides, and/or CL285032; TPEN, LI171, 6-hydroxy-DL-DOPA; or N-methyl-N-[3-(3-methyl[1,2,4]triazolo[4,3-b]pyridazin-6-yl)phenyl]acetamide; or, an enantiomer or stereoisomer thereof, which in alternative embodiments can comprise one or more substituent and/or group replacements with a substituent and/or group having substantially similar physical or chemical properties which produce substantially similar biological properties to compounds used to practice methods or uses as provided herein. In one embodiment, the purpose of exchanging one bioisostere for another is to enhance the desired biological or physical properties of a compound without making significant changes in chemical structures.

For example, in one embodiment, one or more hydrogen atom(s) is replaced with one or more fluorine atom(s), for example, at a site of metabolic oxidation; this may prevent metabolism (catabolism) from taking place. Because the fluorine atom is similar in size to the hydrogen atom the overall topology of the molecule is not significantly affected, leaving the desired biological activity unaffected. However, with a blocked pathway for metabolism, the molecule may have a longer half-life or be less toxic, and the like.

### Products of Manufacture and Kits

Provided are products of manufacture and kits comprising ingredients or components for practicing methods as provided herein, for example, comprising anti-Lin28 nucleic acids and polypeptides, and/or CL285032; TPEN, LI171, 6-hydroxy-DL-DOPA; or N-methyl-N-[3-(3-methyl[1,2,4]triazolo[4,3-b]pyridazin-6-yl)phenyl]acetamide; or, an enantiomer or stereoisomer thereof, and for example, for use in: removing cardiac cells impaired or functionally diminished by stress or age; initiating, inducing or accelerating a cardiac muscle repair or tissue regeneration, a cardiac vasculature repair or tissue regeneration or a cardiac connective tissue repair or tissue regeneration; treating or ameliorating age-related or stress-related cardiomyopathy; and/or treating or ameliorating a heart injury or an injury subsequent to a myocardial infarction (MI). In alternative embodiments, provided with the products of manufacture and kits are instructions for practicing methods as provided herein.

The invention will be further described with reference to the following examples; however, it is to be understood that the invention is not limited to such examples.

### Example 1: Surface Lin28A Expression Consistent with Cellular Stress Parallels Indicators of Senescence

The following example describes how surface Lin28A expression is consistent with cellular stress and parallels indicators of senescence.

This study reveals a novel role for Lin28A in cardiac interstitial cells (CICs) corresponding to surface expression in response to oxidative stress conditions such as those typically present in damaged or aged myocardium. The relationship of surface Lin28A to stress response of CICs including polyploidization, reduced proliferation, and senescence reveals surface Lin28A as a novel marker of cellular senescence. Surface Lin28A expression serves as a phenotypic marker for CICs possessing characteristics of aged cells. Lin28 shift from intracellular to extracellular expression in conjunction with oxidative stress in this study advances understanding of molecular events in the aging myocardium.

### Methods

### Mouse Non-Myocyte Cardiac Cell Isolation and Culture

Mouse non-myocyte cardiac cells (cardiac interstitial cells, CICs) were isolated from both male and female adult FVB mice and maintained as previously described.¹⁰ FVB CICs were evaluated as fresh isolates and culture, primarily at passage 2, with all experiments conducted below passage 10. Cultured CICs were maintained in 5% CO2 and either 1 or 21% O2. Cells were plated and passaged at the same density and media was changed every three days.

### Immunohistochemistry

Mouse hearts were retroperfused, removed from the animal and fixed in 10% formalin overnight. Tissue was then treated with 70% ethanol prior to paraffin embedding using a Leica eASP300 enclosed tissue processor (Leica Biosystems, Buffalo Grove, IL). Paraffin processed tissues were cut into 5-micron section and slide mounted using a HM 355S Automatic Microtome (Thermo Fisher Scientific, Waltham, MA). Heart sections were deparaffinized and incubated with primary and secondary antibodies as previously described.¹⁰ Primary antibody Lin28A was applied 1:200 (PA1-096; Thermo Fisher Scientific, Waltham, MA) overnight in 4 degree fridge and secondary antibody 1:400 (Thermo Fisher Scientific, Waltham, MA) antibodies for 1.5 hours room temperature. The tissue is then rinsed with PBS, DNA is stained with 1 nM DAPI (ThermoFisher, Carlsbad, CA) 1:10,000 in PBS for 10 minutes and Vectashield mounting medium is applied (Vector Labs, Burlingame, CA). Slides were visualized using a Leica TCS SP8 confocal microscope.

### Immunocytochemistry

Total CICs were placed at a density of 10,000 per well of a two-chamber permanox slide and grown for 48 hours. For Lin28 surface analysis, live cells were incubated with Lin28A (1:20) in wash buffer in the incubator for 30 minutes. Cells were washed and labeled with secondary antibody (1:50) in the incubator for 30 minutes. Cells were then fixed in 4% paraformaldehyde for 10 minutes at room temperature. Cells are then permeabilized using 0.03M glycine for 5 minutes followed by 0.5% Triton-X 100/PBS for 10 minutes to reduce non-specific binding. Cells were blocked with 10% horse serum in PBS for 30 minutes at room temperature followed by primary antibody in 10% horse serum in PBS overnight at 4° C. The next day, cells were rinsed with PBS then labeled with secondary antibody and/or phalloidin in 10% horse serum in PBS for 45 minutes. Cells are then rinsed with PBS, DNA is stained with 1 nM 4′,6-diamidino-2-phenylindole (DAPI) (ThermoFisher, Carlsbad, CA) 1:10,000 in PBS for 5 minutes and Vectashield mounting medium is applied (Vector Labs, Burlingame, CA). Slides were visualized using a Leica TCS SP8 confocal microscope.

### Immunoblot Sample Preparation and Experiment

CSCs samples were collected in 1× sodium dodecyl sulfate (SDS) sample buffer with protease and phosphatase inhibitors. Cell lysates were boiled for 10 minutes and stored at −80° C. Proteins were loaded into 4-12% Bis-Tris gel (Thermo Fisher Scientific, Waltham, MA) and run in 1×MES SDS running buffer (Thermo Fisher Scientific, Waltham, MA) at 150 V for 1.5 hours on an electrophoresis apparatus (Invitrogen, Carlsbad, CA). Separate proteins were transferred to a polyvinylidene difluoride membrane in 1× transfer buffer (Thermo Fisher Scientific, Waltham, MA) then blocked with Odyssey Blocking Buffer (TBS) (LI-COR, Lincoln, NE) for 1 hour. After blocking, membrane was incubated with primary antibody Lin28A (1:100) and GAPDH (1:200) in Odyssey Blocking Buffer at 4° C. overnight. The next day, the membrane was washed with 1×TBST 3 times at 15 minutes, room temperature on an orbital rocker. The membrane was then incubated with secondary antibodies for Lin28A (1:500) and GAPDH (1:1000) in Odyssey Blocking Buffer for 1.5 hours at room temperature on an orbital rocker followed by three washes of 1×TBST for 15 minutes a wash, room temperature on an orbital rocker. The membrane was then scanned using an Odyssey CLx (LI-COR, Lincoln, NE).

### Flow Cytometry

To prepare CICs for AMNIS IMAGESTREAM™, approximately 50,000 cells were disassociated if needed, suspended in media, then centrifuged at 1300 rpm for 5 minutes at 22° C. For Lin28A surface analysis, live cells were suspended and incubated with Lin28A (1:20) in wash buffer (0.5% BSA/PBS) on ice for 30 minutes. Cells were washed and resuspended with secondary antibody (1:50) on ice for 30 minutes. Cells were either sorted or then fixed in 4% paraformaldehyde. Following fixation, cells were either incubated with senescence associated b-galactosidase according to manufacturer recommendations (CELLEVENT SENESCENCE GREEN FLOW CYTOMETRY ASSAY™, ThermoFisher), or with 1 nM 4′,6-diamidino-2-phenylindole (DAPI) at 1:20,000 for ten minutes. Cells were washed and imaged with flow cytometry quantitation on a LUMINEX™ AMNIS IMAGESTREAM™ using the AMNIS INSPIRE™ software. Data was analyzed using AMNIS IDEAS™ software (Luminex).

### Hydrogen Peroxide Analysis

Hydrogen peroxide is a known reagent to cause oxidative stress and is a technique commonly used by our team (PMID: 31902324). To confirm surface Lin28A is involved in a response to oxidative stress, passage 2 CICs that were maintained in 1% oxygen were plated in a 6-well plate at a density of 50,000 cells per well in regular media overnight. After twenty-four hours, cells were then washed and media was changed to either regular media or low serum without growth supplements. After another twenty hours, samples in low serum without growth supplements had media changed to low serum without growth supplements and 300 QM Hydrogen Peroxide for four hours. Live cells were then rinsed, suspended and incubated with Lin28A (1:20) in wash buffer (0.5% BSA/PBS) on ice for 30 minutes then washed and resuspended with secondary antibody (1:50) on ice for 30 minutes. Cells were washed and imaged with flow cytometry quantitation on a LUMINEX™ AMNIS IMAGESTREAM™ using the AMNIS INSPIRE™ software. Data was analyzed using AMNIS IDEAS™ software (Luminex).

### ROS and ROS/RNS Analysis

Reactive oxygen species (ROS) and reactive nitrogen species (RNS) are a contributing factor leading to accelerated aging.³⁸⁻⁴² To measure ROS and ROS/RNS activity, cells from 1% and 21% oxygen content were plated at passage 1 split on a 96 well plate at a density of 500 cells per well and left to settle overnight. The following day, cells were stained with a cell-permeable fluorogenic probe 2′,7′-Dichlorodihydrofluorescin diacetate (DCFH-DA), which is diffused into cells, oxidized by ROS, and produces a fluorescence intensity proportional to ROS within the cell cytosol, as described by manufacturers (Cell Bio Labs, San Diego, CA). Similarly, ROS/RNS can be measured in a similar fashion with the probe dichlorodihydrofluorescin DiOxyQ (DCFH-DiOxyQ), (Cell Bio Labs, San Diego, CA). Assays were performed according to manufacturer recommendations and measurements were taken using a spectrometer.

### Antioxidant Treatment

Total CICs were isolated from 6-month-old FVB mice and were immediately plated at the same density in either regular media or regular media with Trolox (Sigma-Aldrich, Burlington, MA) in a dose dependent manner. Media, with or without Trolox, was changed and cells were expanded according to standard protocol for two passages, with replating at the same density. At passage 2, Total CICs were then studied for oxidative stress-related properties including proliferation, cell size, surface Lin28A, senescence associated b-gal, ploidy, and nucleation.

### Single-Cell RNA-seq

Freshly isolated single cell suspensions were loaded on a CHROMIUM™ CONTROLLER™ (10× Genomics) and single-cell RNA-Seq libraries were prepared using CHROMIUM™ Single Cell 3′ Library & Gel Bead Kit v3 (10× Genomics; Item 1000075) following manufacturer's protocol. Concentration and fragment size distribution of each library were tested with BIOANALYZER™ (Agilent High Sensitivity DNA Kit, Cat. #5067-4626; average library size: 450-490 bp). The sequencing libraries were quantified by quantitative PCR (KAPA BIOSYSTEMS LIBRARY QUANTIFICATION KIT™ for Illumina platforms P/N KK4824) and Qubit 3.0 with dsDNA HS ASSAY KIT™ (Thermo Fisher Scientific). Sequencing libraries were submitted to the UCSD IGM Genomics Core for sequencing (NovaSeq 6000).

### Single Cell Selection and Quality Control

The raw data was processed with the Cell Ranger pipeline (10× Genomics; version 3.0.1, SF 1). Sequencing reads were aligned to the 10× mouse genome mm10. Cells with fewer than 200 genes were filtered out to avoid inclusion of empty droplets in downstream analysis. Based on UMI and gene detection distribution droplets multiplets were excluded using the Interquartile Range Rule (values over the third quartile and 1.5 the interquartile range are considered outliers). Cell with more than 15% of mitochondrial gene UMI count and genes detected in fewer than three cells were filtered out using Seurat R Package (v4.0.2).⁴³ The first 8 principal components were found to be significant to perform dimensionality reduction. Preparations derived from Lin28 cell surface sorting yielded 7086 barcoded cells for analysis, from which 4334 corresponded to Lin28^{S-}, and 2752 corresponded to Lin28^{S+} (874 Lin28^{S+}/cKit−, 1878 Lin28^{S+}/cKit⁺). Final removal of unwanted sources of variation and batch effect corrections was performed using Seurat R Package (v4.0.2).⁴³

### Dimensionality Reduction and Unsupervised Clustering

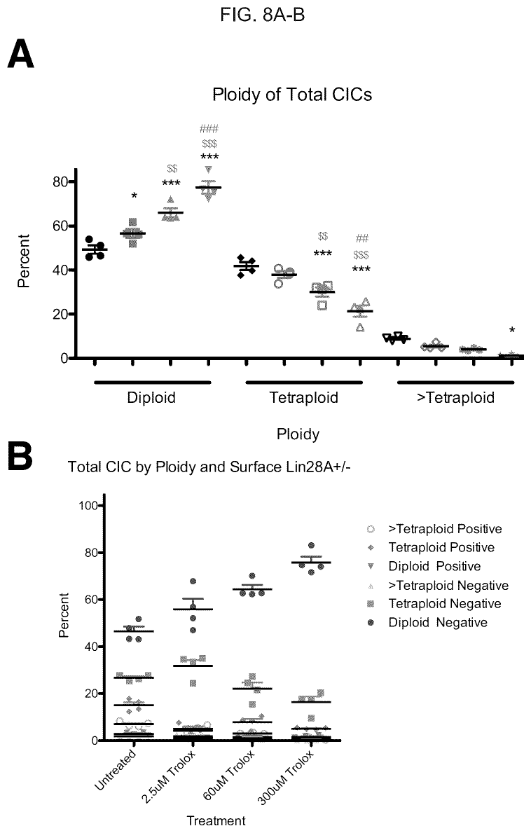

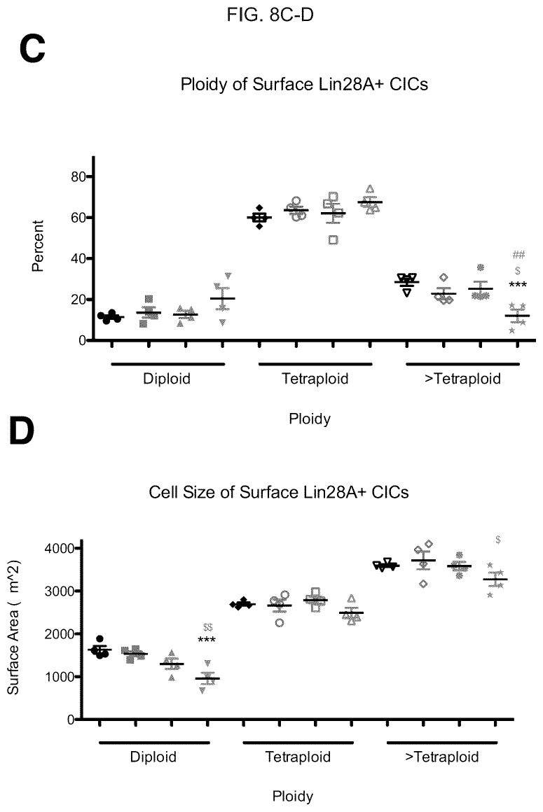

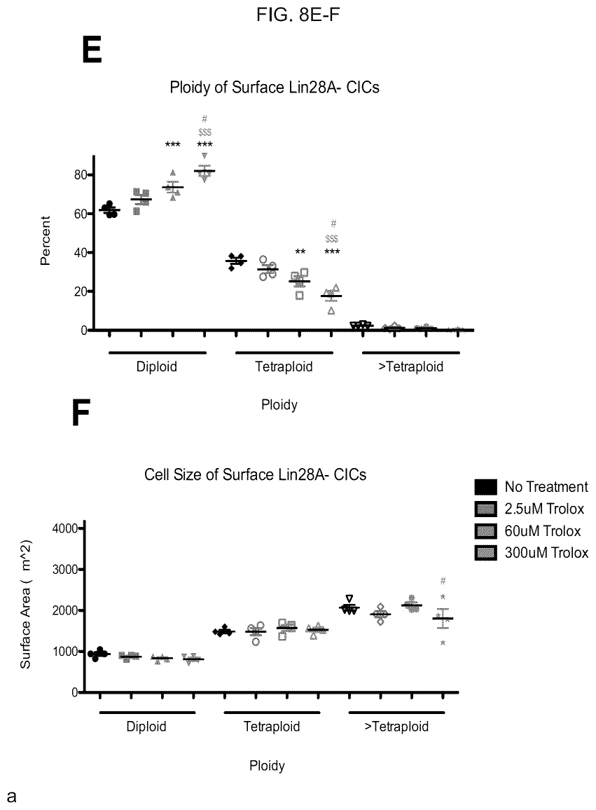

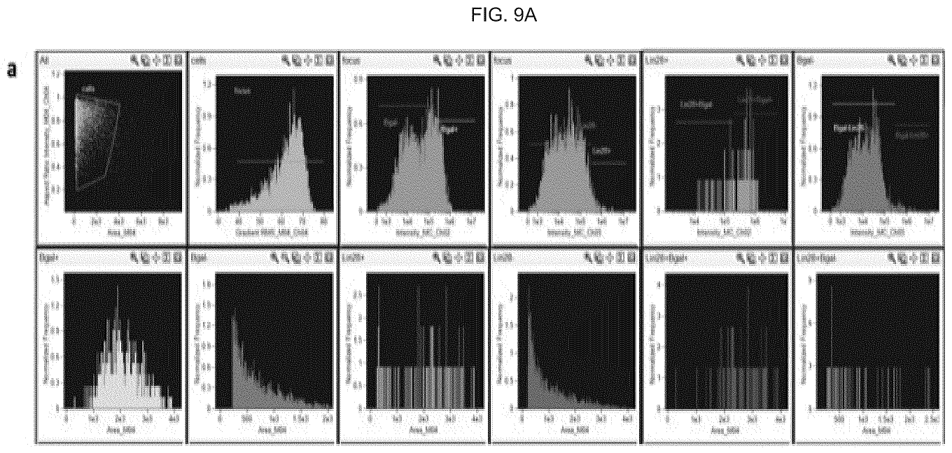

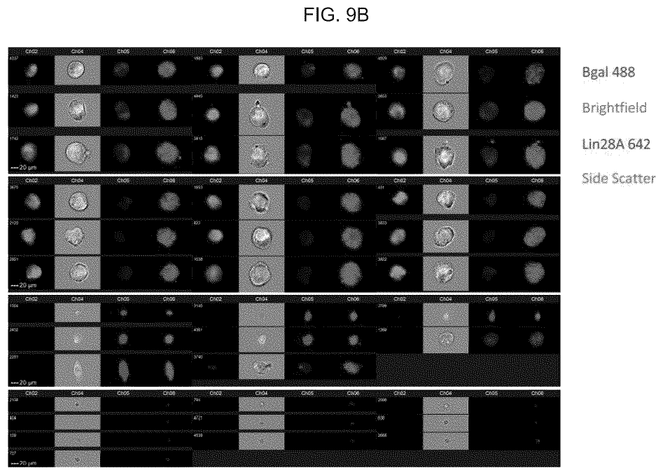

Approximately 2000 variable genes were selected based on their expression and dispersion. Prior dimensionality reduction, data was scaled to mean expression equal to 0 and variance across cells equal to 1. Principal component analysis was performed on the scaled data as a linear dimensionality reduction approach. The first 8 most significant principal components (PCs) were selected for non-linear dimensional reduction (PCA, tSNE and UMAP; Supp. FIG. 8) and unsupervised clustering using complementary methods, including supervised PC selection, Jackstraw statistical and heuristic approaches. Cluster were validated by concurrent expression of housekeeping genes (Gapdh, Actb, Rplp0, B2m and Ywhaz; Supp. FIG. 9). Cell type annotation of unsupervised clusters was based on cell known markers. Clusters not expressing canonical cell markers were classified using the clustifyr package⁴⁴ using the Tabula muris data as reference set for classification.⁴⁵

### Differential Expression Analysis

Differential expression analysis was done using Wilcoxon rank sum test and selecting for a threshold of 0.05 for an adjusted p-value and a log (FC)>0.25 was used to define statistically significant and differentially expressed genes (DEGs).

### Gene Ontology Analysis

Gene ontology (GO) enrichment analysis for DEGs lists derived from Neonatal, LVAD and Intermediate cells was performed using the enrich Gene ontology (GO) and compare Cluster functions of clusterProfiler (3.16.1) R package.⁴⁶ GO terms were selected with p-value cutoff of 0.05 using BH method.

### Data Availability

scRNA-Seq data generated in this study has been uploaded to the Gene Expression Omnibus (GEO) database (GSE186176, released Oct. 22, 2021).

### Animal Experiments

All animal protocols were approved by the Institutional Animal Care and Use Committee of San Diego State University and conform to the Guide for the Care and Use of Laboratory Animals published by the US National Institutes of Health. Adult mice aged 6 months from gender matched mice of the same strain (FVB) were used for the study.

### Statistical Analyses

All data are expressed as mean+/−standard error of mean. Statistical analyses were performed within and between group comparisons, student t-test, one- and two-way ANOVA was applied with Bonferroni post-test, when applicable. using Graph PAD PRISM V5.0™ (GraphPad Software, La Jolla, CA). A value of less than 0.05 was considered statistically significant (* p<0.05, ** p<0.01, *** p<0.001).

### Results

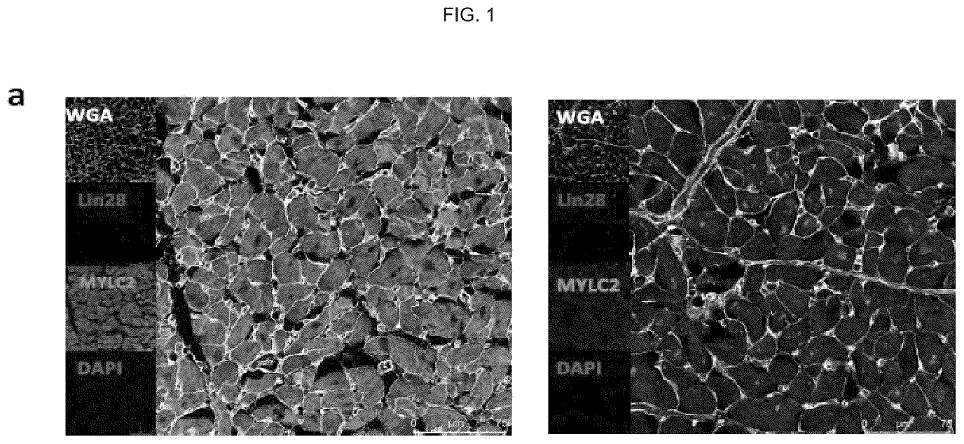

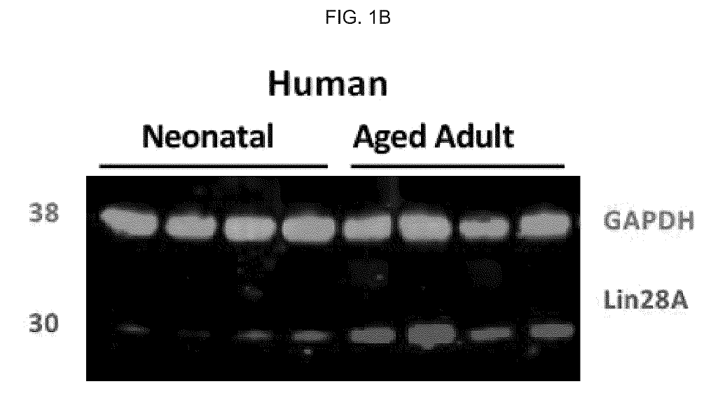

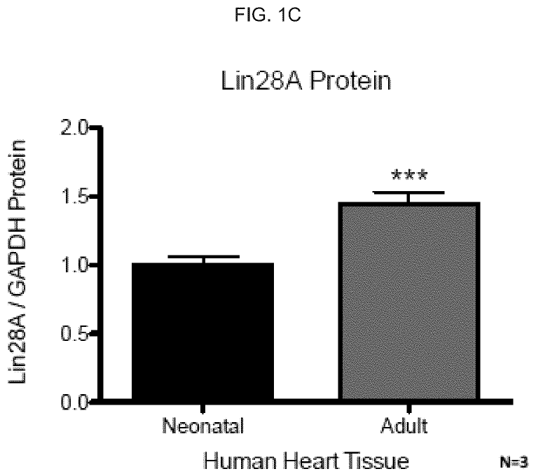

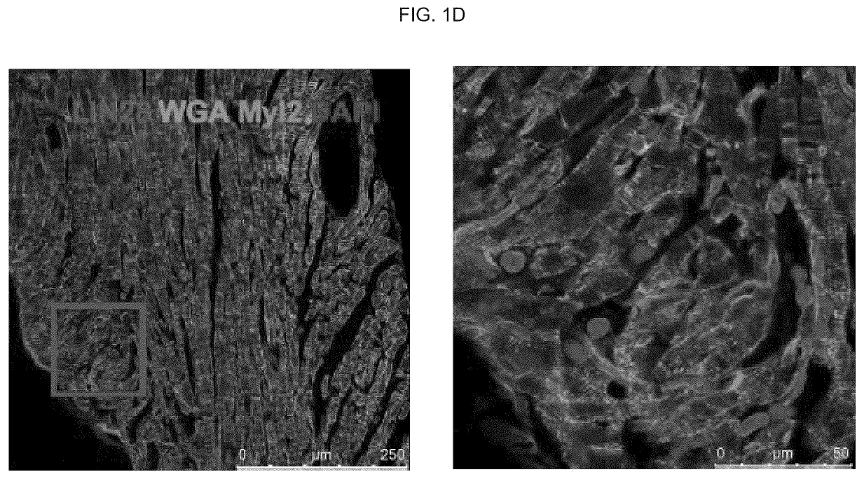

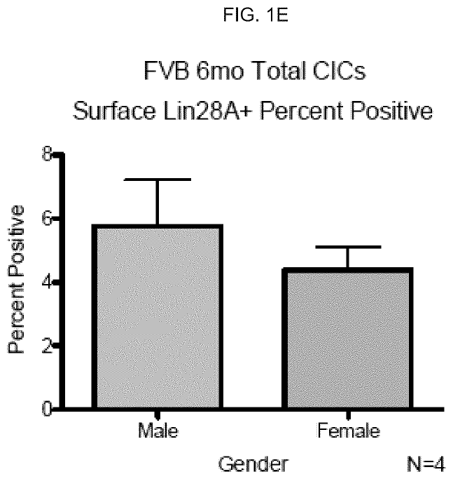

Lin28A is expressed in human and rodent cardiac tissue. Normal neonatal and adult ventricle cardiac tissue was immunolabeled for Lin28A, myosin light chain, connective tissue, and DNA. CICs and cardiomyocytes express Lin28A in adult normal and heart failure samples, whereas fetal tissue demonstrated fewer Lin28A positive stained cells (FIG. 1a). Lin28A protein level was significantly increased in aged adult (1.445±0.2767), compared to control neonatal (1.00±0.1898) samples (FIG. 1b; p<0.001). Heart tissue sections from adult mice showed comparable Lin28A immunoreactivity (FIG. 1c). Total CICs were isolated from ventricle tissue of adult FVB mice of both male and female mice in 4 experiments, stained for surface Lin28A in fresh, live cells and sorted. Surface Lin28A expression between male (5.760±2.916) versus female (4.393±1.427) samples showed no statistical differences (FIG. 1d).

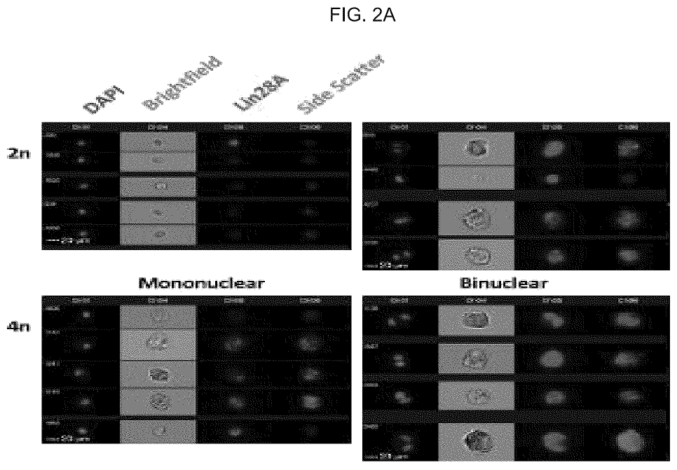

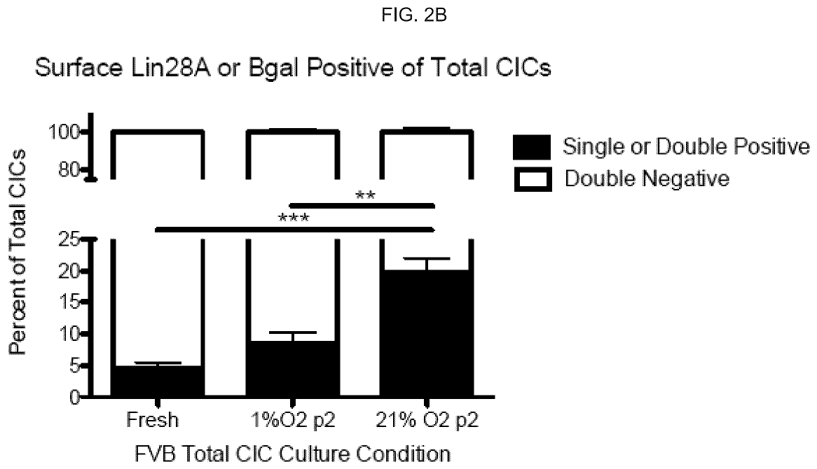

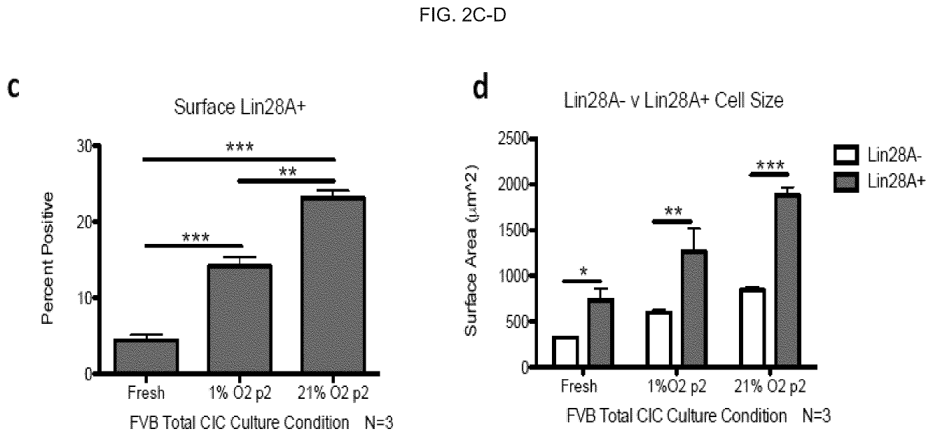

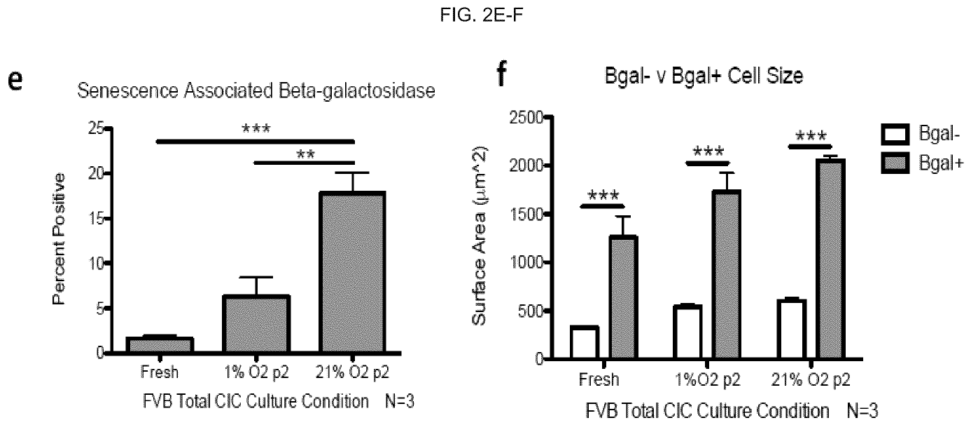

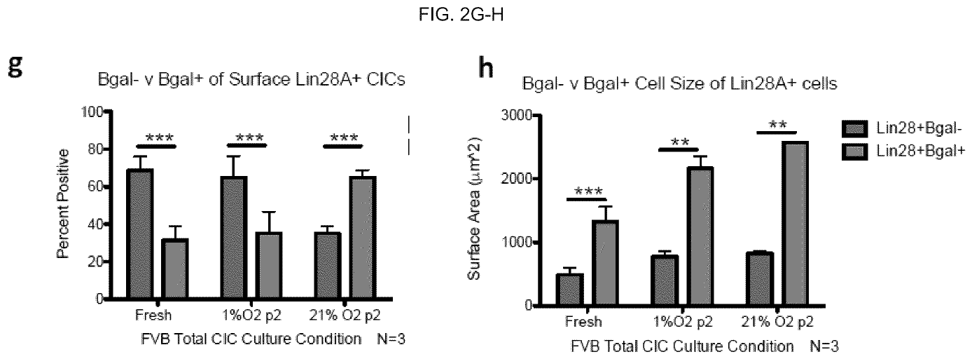

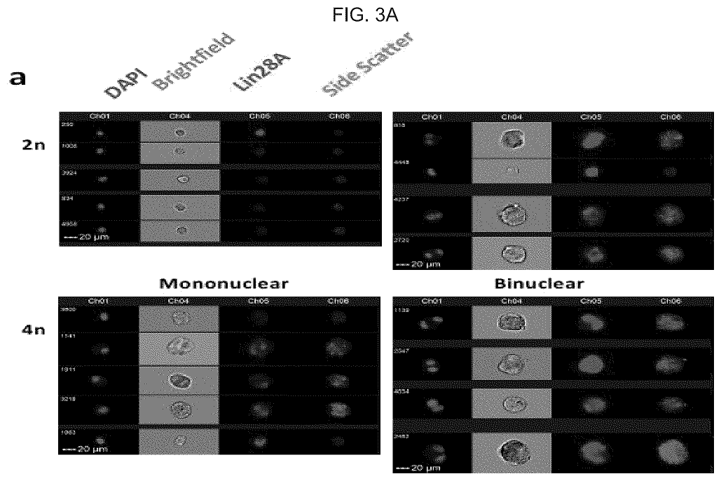

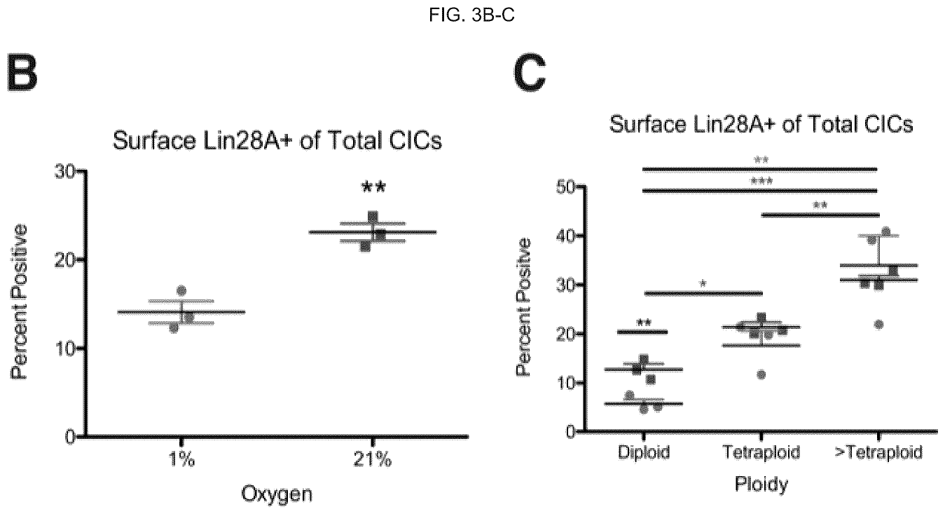

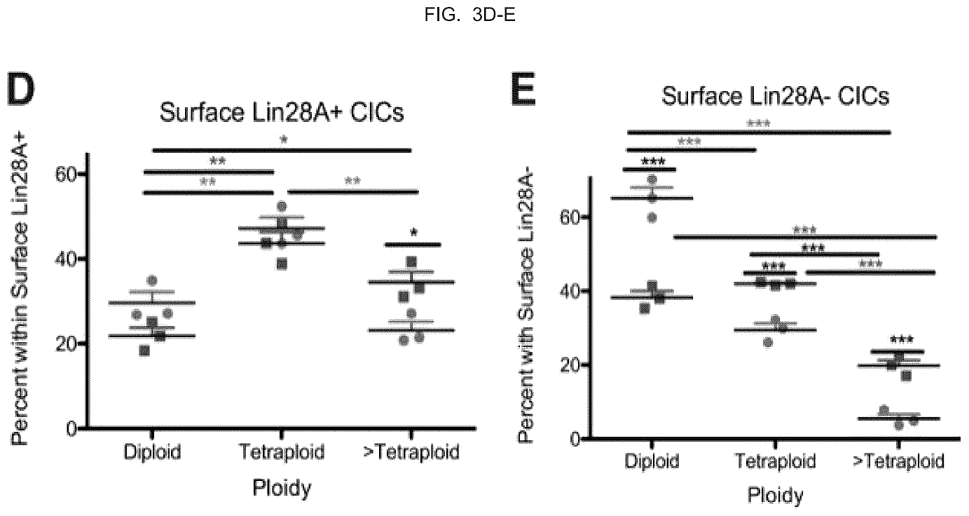

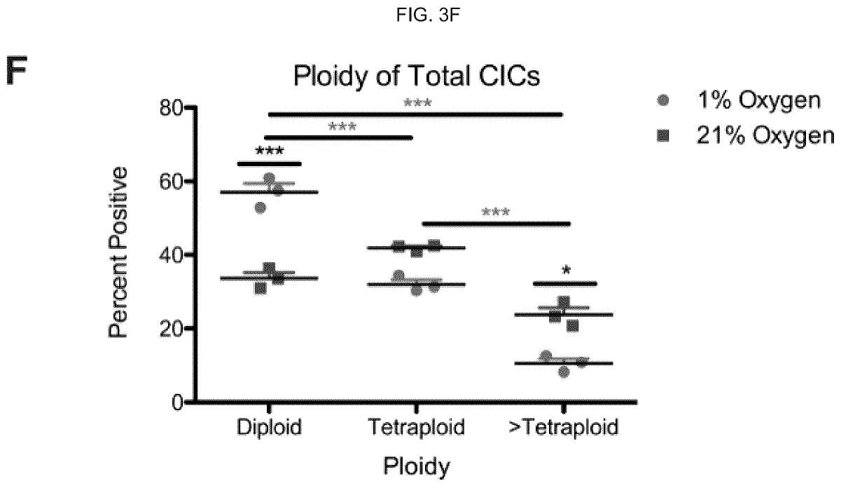

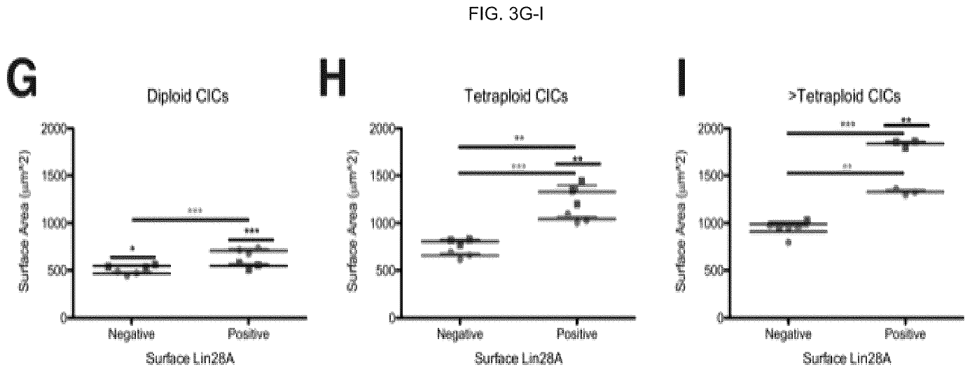

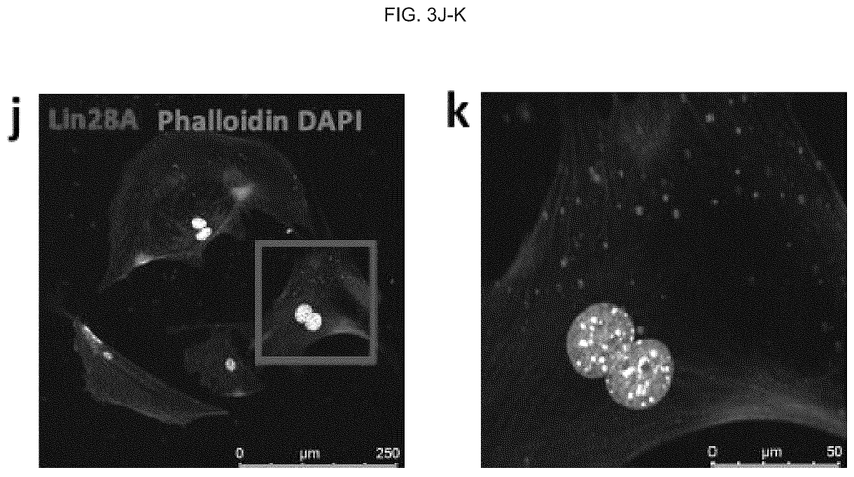

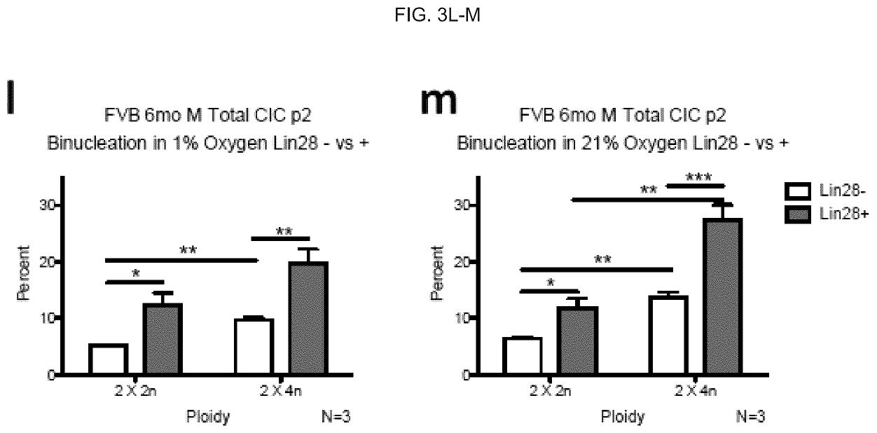

Oxidative stress promotes Lin28A surface expression together with CIC senescence phenotype Surface Lin28A(+) (Lin28A^{S+}) expression was assessed relative to senescence-associated b-galactosidase (b-gal⁺) as well as cell area. Analyses were performed using initial isolates of CICs or descendants minimally expanded in vitro by two passages in either 1% or 21% O₂. AMNIS IMAGESTREAM™ was used for flow cytometry visualization of CIC phenotypic characteristics in population-based analyses (FIG. 2a, Supp. FIG. 1). Oxidative stress increased frequency of Lin28A^{S+} or b-gal⁺ CICs expanded at 21% O₂ level (19.7% 2.4) versus cells at 1% O₂ (8.4%±1.8) or freshly isolated CICs (4.5%±1.0) (FIG. 2b; **p<0.01). In vitro culture increased Lin28A^{S+} expression at 21% O₂ (21.1%±1.7) versus either 1% O₂ (14.1%±0.2.2) or CICs direct from tissue without culture (4.4%±1.4) (FIG. 2c; **p<0.01). The role of oxidative stress in promotion of Lin28A^{S+} was confirmed by treatment with hydrogen peroxide (H₂O₂), which increased frequency of Lin28A^{S+} CICs when expanded in 1% oxygen (Supp. FIG. 2). Cell area of Lin28A^{S+} was consistently larger compared to Lin28A^{S-} CIC whether assessed using freshly isolated (734.5±258 μm² versus 324.6±9.138 μm² respectively) or cells cultured under oxygen levels of 1% (1261±439.7 μm² versus 594.7±55.7 μm², respectively) and 21% (1879±182.7 μm² versus 844.1±67.0 μm², respectively) (FIG. 2d; *p<0.05). Similarly, b-gal⁺ CICs were more prevalent at 21% O₂ (17.78±4.7) relative to expansion in 1% O₂ (6.30±3.7) or in fresh isolates (1.640±0.6398) (FIG. 2e; **p<0.01). Likewise, surface area of b-gal⁺ versus b-gal⁻ CICs was highest in 21% O₂ (2050□94.5 versus 601.0±56.8), lower in 1% O₂ (1732±330.8 versus 538.3±44.4), and lowest in fresh isolates (1256±445.9 versus 328.0±12.90) (FIG. 2f; ***p<0.001). Lin28A^{S+} CICs demonstrated senescence-associated characteristics of b-gal⁺ and increased cell area coincident with oxidative stress from culture in 21% O₂ (FIG. 2g; ***p<0.001). The majority of CICs from freshly isolated or 1% O₂ culture were Lin28A^{S+}/b-gal⁻ (68.65%±14.8 and 64.83%□19.9, respectively). In contrast, a majority of CICs (64.9%±6.9) from 21% O₂ cultures were Lin28A^{S+}/b-gal⁻. CIC surface area consistently increases with oxidative stress from culture conditions (FIG. 2h; **p<0.01). Comparison of Lin28A^{S+}/b-gal⁺ versus Lin28A^{S+}/b-gal⁻ shows larger cell area whether in fresh (1328 μm²±459.8 versus 487.7 μm²±221.4), 1% O₂ (2167 μm²±322.8 versus 767.6 μm²±147.6), or 21% O₂ (2571 μm²±18.32 versus 824.3 μm²±63.93). The causal role for oxidative stress promoting Lin28A^{S+} was also demonstrated in the total unfractionated CIC population treated with H₂O₂ (Supp FIG. 3).

Additional analyses were undertaken to delineate whether acquisition of senescence characteristics of b-gal⁺ and/or cell area enlargement influence Lin28A^{S+}. Lin28A^{S-}/b-gal⁺ CICs were absent from freshly isolated CICs with an increasing percent of Lin28A^{S+}/b-gal⁺ CICs in 1% and 21% O₂ culture (5.3±1.9% and 15.3□2.0%, respectively) (Supp. FIG. 3a; ***p<0.001). Lin28A^{S-}/b-gal⁺ CICs exhibit dramatic increases in surface area compared to Lin28A^{S-}/b-gal⁻ in both 1% O₂ (1645 μm²±191.9 versus 533.1 μm²±25.8) and 21% O₂ (1977 μm²±53.62 versus 613.6 μm²±29.14) (Supp FIG. 3b; ***p<0.001).

In reviewing b-gal⁺ CICs, results show fresh isolates are rare for Lin28A^{S+} and are less frequently positive, compared to Lin28A^{S-} in 1% O₂ (84.23±3.06 versus 15.51±3.284) and 21% O₂ (89.38±1.412 versus 10.28□1.363) (Supp FIG. 3c; ***p<0.001). Surface area of b-gal⁺ CICs are slightly larger when also positive for Lin28A^{S+}, compared to Lin28A^{S-}, in 1% O₂ (2172 μm²±185.6 versus 1651 μm²±177.4) and 21% O₂ (2523 μm²±48.58 versus 2011 μm²±43.37) (Supp FIG. 3d; *p<0.05). In reviewing b-gal⁻ CICs, results show CICs are rarely Lin28A^{S+} in fresh isolates (4.138□0.6901) and CICs at passage 2 and expanded in 1% (2.147±0.5949) oxygen and 21% (1.515±0.0834) oxygen (Supp FIG. 3e; ***p<0.001). Surface area of b-gal⁻ CICs are consistently small regardless of being positive for Lin28A^{S+}, compared to Lin28A^{S-}, in fresh isolates (669.2±121.3 versus 325.1±4.102), 1% O₂ (615.4±29.52 versus 533.3±25.18) and 21% O₂ (858.8±63.29 versus 778.9±95.33) (Supp FIG. 3f; **p<0.01).

Collectively, these results demonstrate acquisition of Lin28A^{S+} in response to oxidative stress that correlates with senescence-associated characteristics of b-gal⁺ and cell area enlargement. Conversely, Lin28A^{S-} CICs exhibit diminished senescence-associated characteristics. Therefore, Lin28A^{S+} expression serves as a marker of CIC senescence.

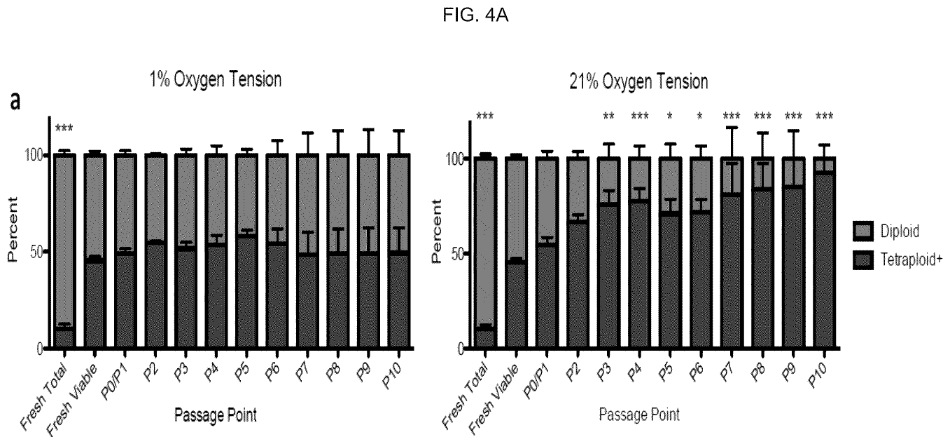

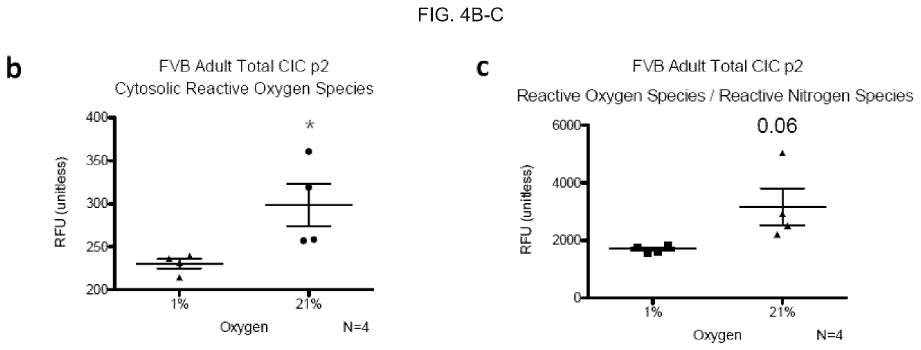

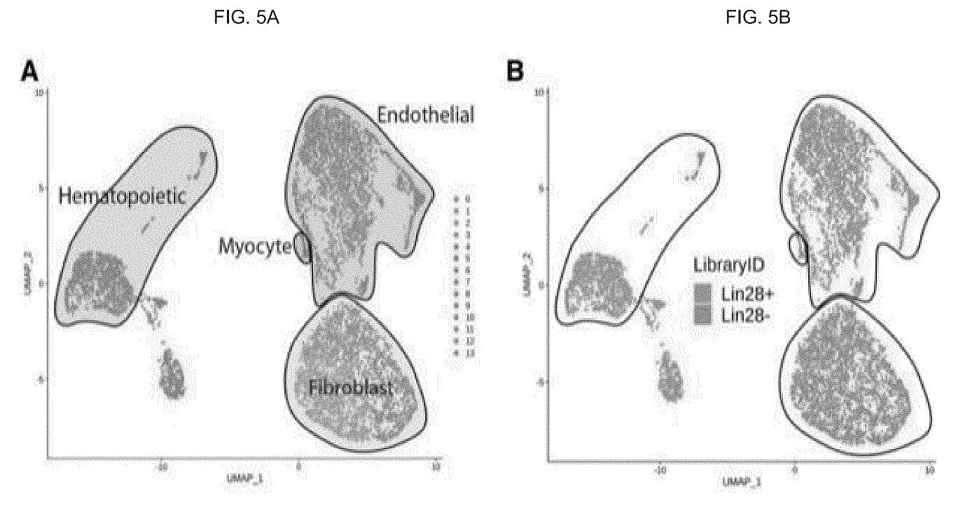

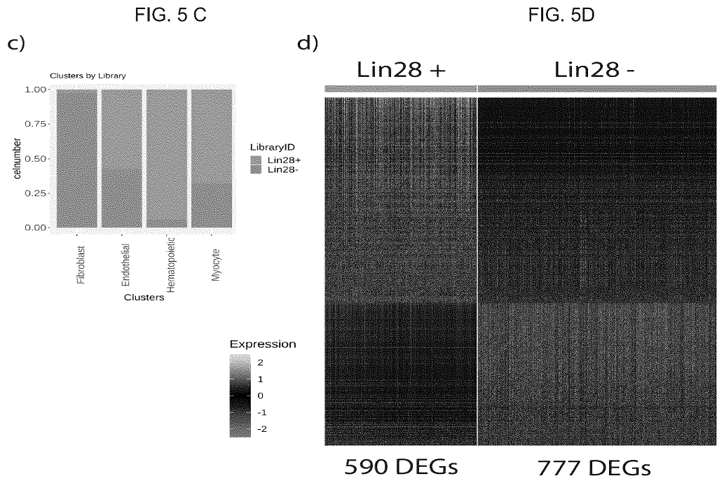

Diploid content favored by low oxidative stress conditions tracks with CIC phenotypes of surface Lin28A(−) and small size. Cellular polyploidization is influenced by environmental stress, can alter cell cycle progression, and has been linked to senescence.³⁰⁻³⁵ Since Lin28A^{S+} in CICs correlates with oxidative stress and senescence markers (FIG. 2), polyploidization was evaluated as an additional phenotype acquired with Lin28A^{S+}. Cellular DNA content was determined using DAPI stain in conjunction with Lin28A^{S+} using AMNIS IMAGESTREAM™ (FIG. 3a, Supp. FIG. 4). Consistent with previous results (FIG. 2), Lin28A^{S+} were prevalent in 21%, compared to 1% O₂ (23.1%+/−1.0 versus 14.1+/−1.3) (see FIG. 3B and FIG. 3C) in the total CIC population (FIG. 3b; **p<0.01). Increased ploidy level correlated with Lin28A^{S+} CIC cells in both 1% O₂ as well as 21% O₂ (FIG. 3c; *p<0.005). Polyploidization of Lin28A^{S+} CICs correlates with higher oxidative stress in culture conditions of 21% O₂ compared to 1% O₂ (FIG. 3d; *p<0.05, Supp. FIG. 5a), whereas diploid CICs were more frequent in 1% O₂ compared to 21% O₂. Furthermore, diploid CICs were predominantly Lin28A^{S-} in 1% O₂ with higher polyploid content associated with Lin28A^{S-} CICs in 21% O₂ (FIG. 3e; ***p<0.001, Supp. FIG. 5b). While diploid CICs were the majority population in 1% O₂ (57.1%±2.3), polyploid CICs prevail in 21% O₂ (66.33%±1.56) (FIG. 3f; *p<0.05). Diploid content correlates with smaller surface area relative to tetraploid CICs regardless of surface Lin28A expression or culture O₂ level (FIGS. 3f and 3g; *p<0.05). Although larger overall, subsets of tetraploid CICs segregate based upon surface Lin28A expression with Lin28A^{S-} cells smaller than their Lin28A^{S+} counterparts (FIG. 3h; **p<0.01). However, greater than tetraploid content results in the largest surface area regardless of surface Lin28A expression or culture conditions (FIG. 3i; **p<0.01). Taken together, these results demonstrate that diploid CICs are favored under culture conditions of low oxidative stress and display a phenotype of Lin28A^{S-} and relatively small cell area.

Binucleation correlates with Surface Lin28A positive CICs. Nucleation level was determined as part of understanding CIC ploidy status since polyploidy occurs in both mononuclear and multinuclear cells.¹⁰ Binucleation is more frequently present in polyploid Lin28A^{S+} compared to Lin28A^{S-} CICs in 1% O₂ cultures (32.0%±3.7 versus 14.8%±0.65, respectively; FIG. 3l; *p<0.05). Similar preponderance of polyploid Lin28A^{S+} versus Lin28A^{S-} CICs was present in 21% O₂ cultures (39.2%□1.35 and 20.1%±0.73, respectively; FIG. 3m; *p<0.05). Thus, multinucleation is more likely when Lin28A is expressed on the surface of polyploid CICs.

Polyploidization of CICs occurs in response to chronic oxidative stress. Causality of oxidative stress conditions to promote diploid to tetraploid conversion was assessed by serial passaging of CICs in either 1% or 21% oxygen. Freshly isolated CIC stained for DNA content and viability are evenly split between diploid or tetraploid live cells. This 50/50 percentage split between diploid and tetraploid CICs was maintained in 1% O₂ for up to ten sequential passages. In contrast, ploidy shifted to almost exclusively tetraploid CICs when subjected to the same passaging protocol in 21% O₂ (FIG. 4a; *p<0.05, Supp. Table 1). Cytosolic reactive oxygen species measured at passage 2 was significantly increased in 21% O₂ compared to 1% O₂ (298.8±25.1 RFU versus 230.5±5.6 RFU) (FIG. 4b; *p<0.05). Ratio of cytosolic reactive oxygen species to reactive nitrogen species was similarly elevated in the 21% O₂ compared to 1% O₂ (3167±640.4 RFU versus 1702±58.09 RFU) (FIG. 4c). These results demonstrate polyploidization as a cellular response to chronic oxidative stress.

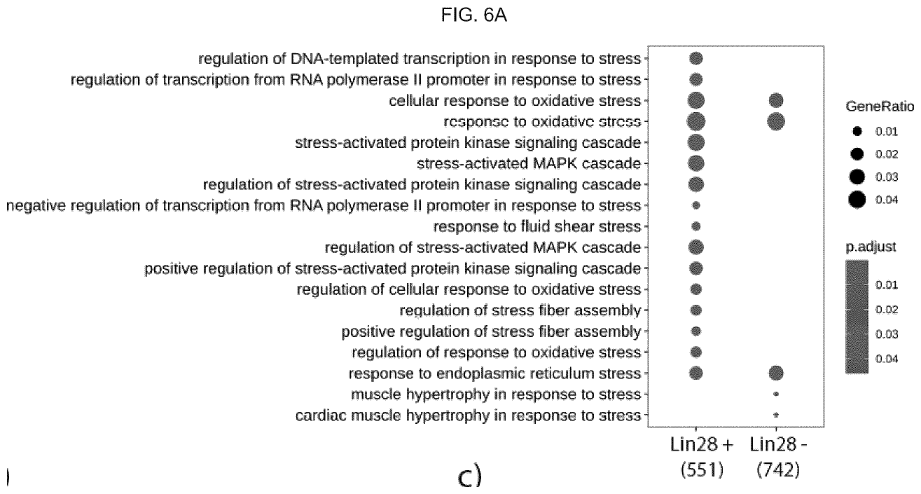

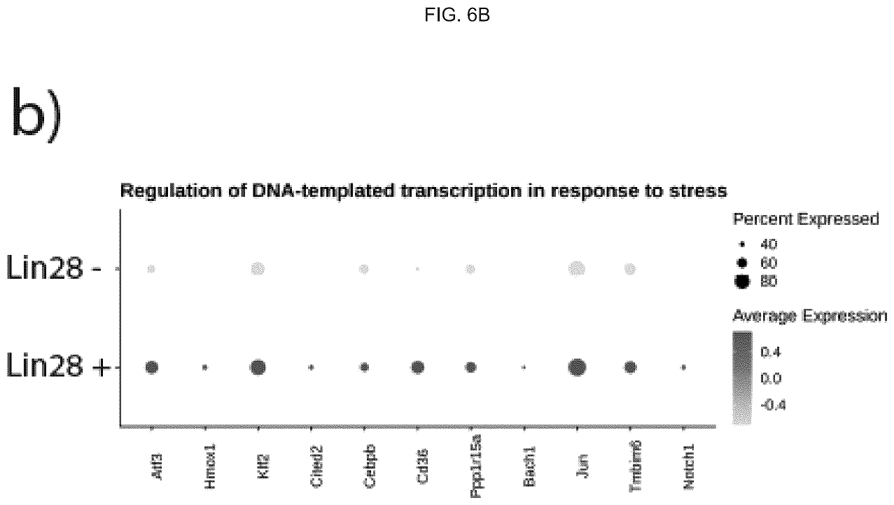

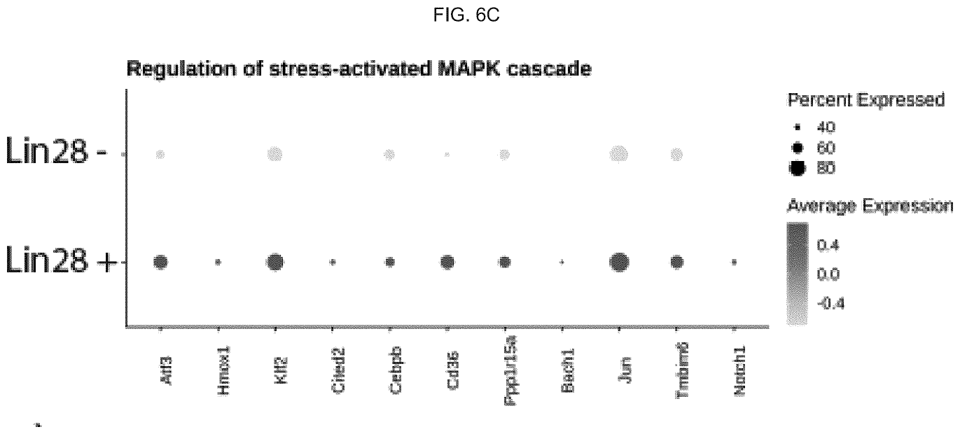

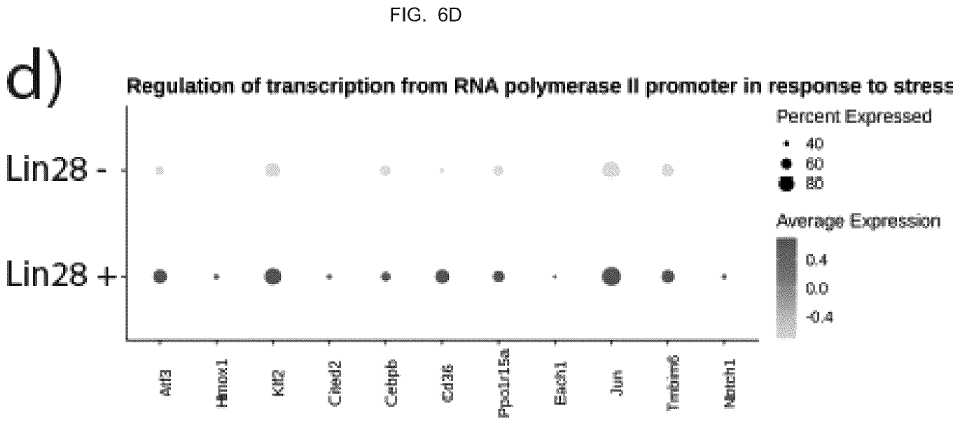

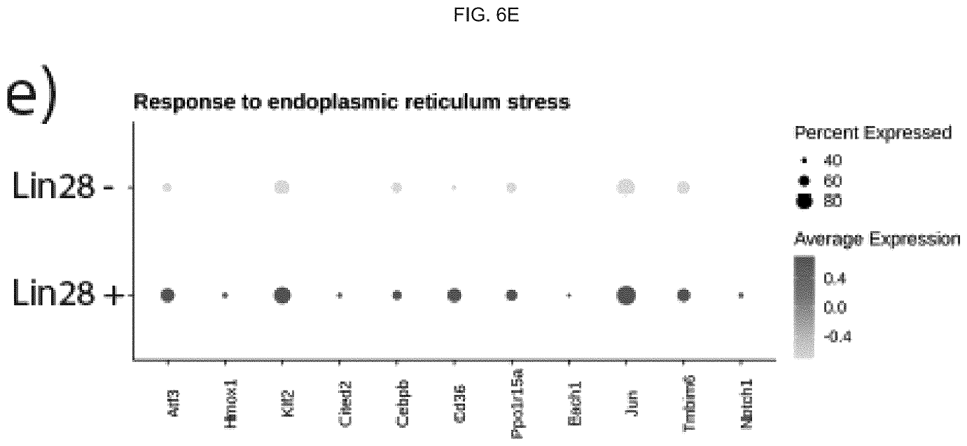

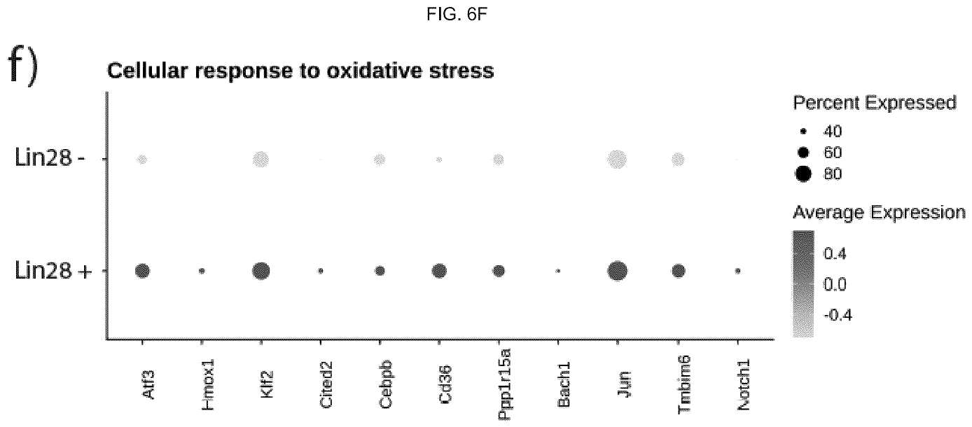

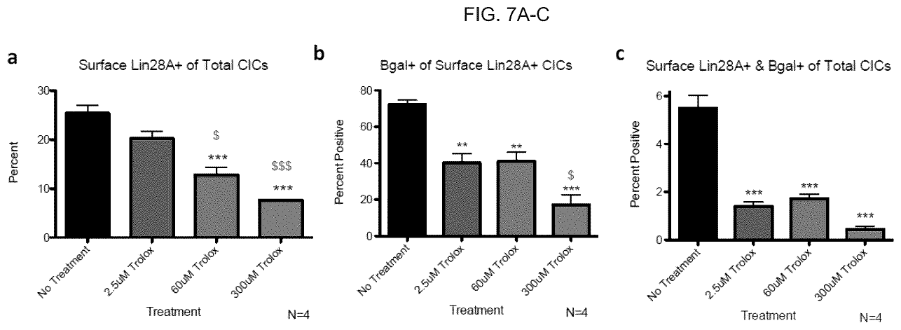

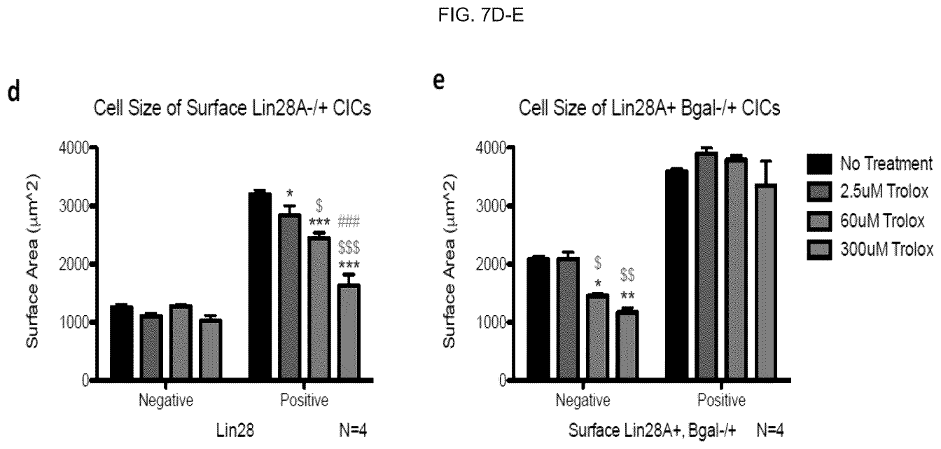

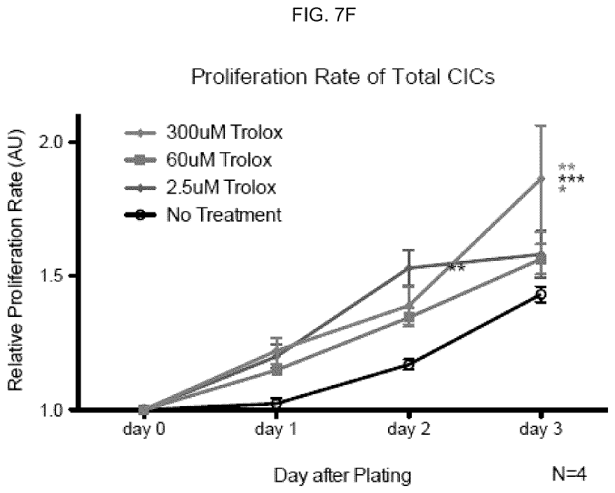

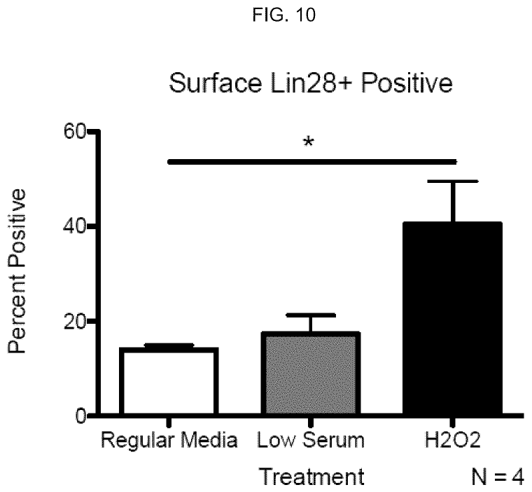

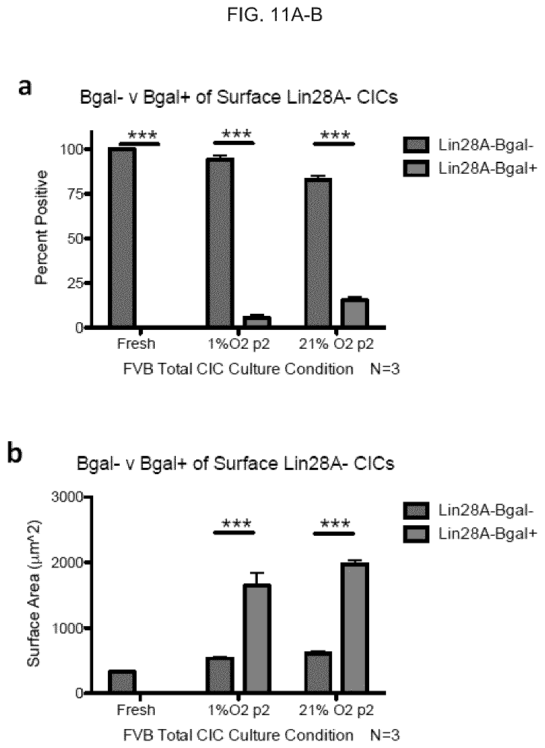

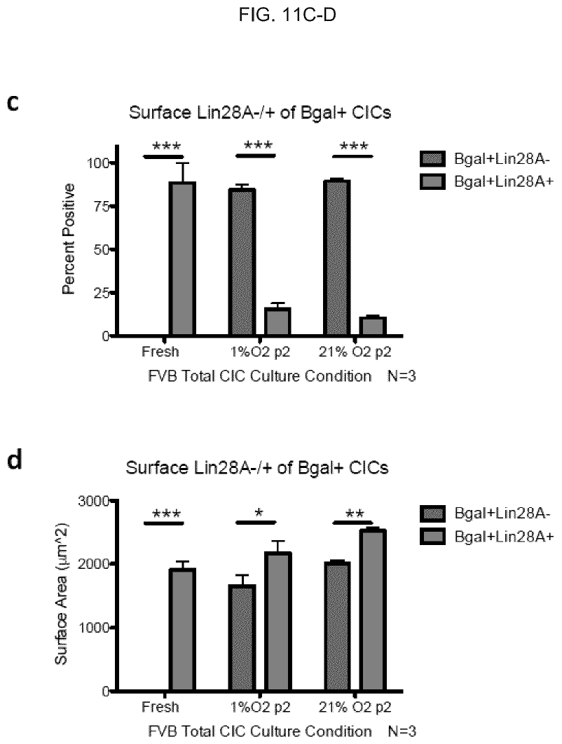

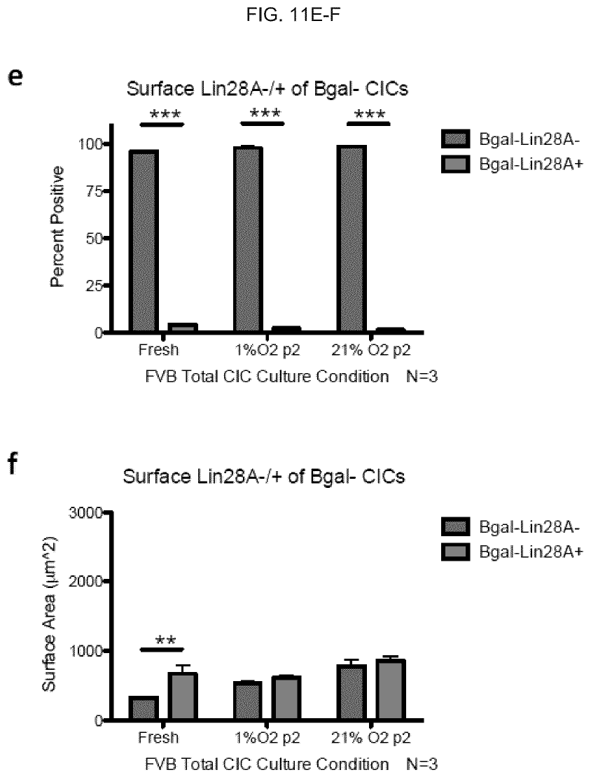

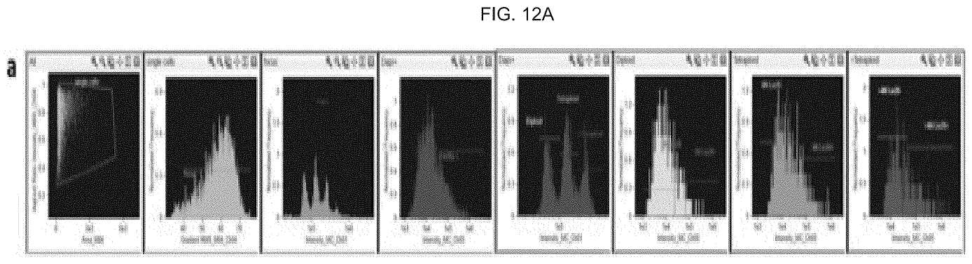

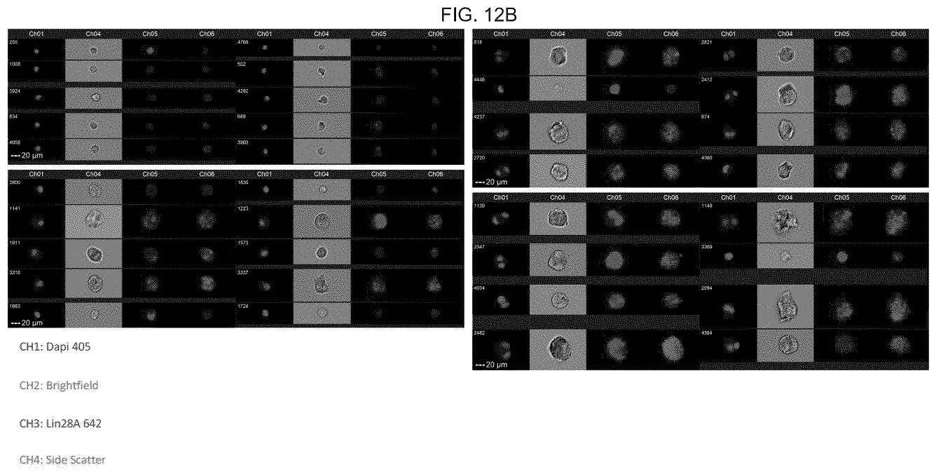

Surface Lin28A CICs are predominantly endothelial and hematopoietic lineage. Single cell RNASEQ evaluated phenotypic differences of CICs based upon surface Lin28 expression presence (Lin28A^{S+}) or absence (Lin28A^{S-}) (Supp. FIG. 6-8). Dimensionality reduction and unsupervised clustering revealed 14 clusters segregated according to transcriptional phenotype (Supp. FIG. 7, 8). Canonical cell markers identified four main cell types in the aggregated libraries: Fibroblast (Clusters 0, 4 and 5), Endothelial (Clusters 1, 2, 7, 11 and 13), Hematopoietic (Clusters 3, 10 and 13) and Myocyte (Cluster 12; FIG. 5a and Supp. FIG. 9). Clusters 6, 8 and 9 did not express markers of the main four cell types. However, these clusters appeared transcriptionally like hematopoietic (Cluster 6 and 9) and endothelial (Cluster 8), consistent with the dimensionality reduction results. The similarity was verified by cross-referencing the transcriptional profile with the reference single cell transcriptome atlas Tabula muris⁴⁷ (Supp. FIG. 10). Normalization to input revealed fibroblast depletion in the Lin28A^{S+} population with an enrichment for endothelial and hematopoietic cell times (FIG. 5b, 5c). Lin28A^{S+} were predominant in clusters 6, 8 and 9 consistent with cell annotation analysis (Supp. FIG. 11). Differential expression analysis revealed transcriptional differences with 590 DEGs identified on Lin28A^{S+} and 777 DEGs on Lin28A^{S-} (FIG. 5d). Together these results confirm inherent differences in both transcriptional phenotypes and cell type distributions in the cardiac interstitial linked to presence of surface Lin28A.

Surface Lin28A CICs upregulate gene markers demonstrating a stress response. DEGs derived from differential expression analysis were used as input for gene ontology analysis. GO term analysis of biological processes revealed an enrichment of various ontologies associated with cellular stress (FIG. 6a). Lin28A^{S+} upregulated genes overlapping with multiple ontologies, such as Atf3, Hmox1, Klf2, Cited2, Cebpb, Cd36, Bach1 and Jun among others (FIG. 6b-6f). Lin28A^{S+} DEGs demonstrated to be involved in the regulation of DNA-template transcription and RNA polymerase in response to stress, stress induce activation of MAPK signaling, response to endoplasmic reticulum and oxidative stress. Collectively these results demonstrate Lin28A^{S+} cells exhibit transcriptional profiles associated with response to cellular stress.

Oxidative stress-induced phenotypic changes in CICs inhibited by anti-oxidant treatment. Beneficial effects of antioxidant treatment to blunt cellular stress as well as cardiomyopathic injury is well documented.⁴⁸⁻⁵¹ Among antioxidant compounds, Trolox is a derivative of vitamin E previously used to lower reactive oxygen species in cultured cells.⁴⁸⁻⁵¹ Therefore, Trolox was used to antagonize oxidative stress in cultures of CICs exposed to chronic high oxidative stress of 21% O₂ incubation conditions. CICs were treated with Trolox in a dose dependent manner (none, 2.5 □M 60□M 300□M) immediately upon plating and throughout outgrowth in 21% O₂ until analysis at passage 2. Surface Lin28A, b-gal, and cell size was analyzed using AMNIS IMAGESTREAM™ and compared to no treatment baseline (Supp Table 2). Frequency of Lin28A^{S+} CICs was reduced after Trolox treatment in a dose dependent manner (FIG. 7a; *p<0.05). Similarly, b-gal⁺ CICs were significantly less prevalent after Trolox treatment, also in a dose dependent manner compared to no treatment baseline (FIG. 7b; *p<0.05). Double positive Lin28A^{S+}/b-gal⁺ CICs were also significantly less frequent after Trolox treatment in a dose dependent manner (FIG. 7c; ***p<0.001). Cell area of surface was significantly decreased for Lin28A^{S+} CICs after Trolox treatment in a dose dependent manner, but cell area of Lin28A^{S-} CICs was unchanged by Trolox treatment (FIG. 7d; *p<0.05). Cell area of Lin28A^{S+} responded differently depending upon b-gal activity, with high dose Trolox promoting smaller cells in b-gal⁻ whereas b-gal⁺ CIC were consistently larger (FIG. 7e; *p<0.05). Proliferation rate remained controlled but was consistently higher when treated with Trolox (FIG. 7f; *p<0.05). Collectively, these results demonstrate inhibition of oxidative-stress mediated changes by Trolox and confirm the parallel relationship between Lin28A^{S+} and senescence-associated characteristics of b-gal⁺ and cell enlargement

Trolox increases diploid population and antagonizes conversion to higher ploidy. Polyploidzation is another hallmark of cellular response to stress and senescence,³⁰⁻³⁵ so additional studies were performed to assess the impact of Trolox upon ploidy state of CICs in relation to surface Lin28A expression when cultured in 21% O₂ (Supp. Table 3). Diploid CICs comprise a significantly higher percent of the total population with Trolox treatment in a dose dependent manner (FIG. 8a; *p<0.05). Trolox treatment reduced the frequency of Lin28A^{S+} in a dose dependent manner (FIG. 8b, Supp. Table 2) demonstrating antagonism of cellular stress. Lin28A^{S+} expression corresponds with increased presence of polyploidy which was reduced by Trolox treatment in a dose dependent manner (FIG. 8c; *p<0.05). Cell size of Lin28A^{S+} CICs correlated with both higher ploidy and larger size, regardless of Trolox dosing (FIG. 8d; *p<0.05). Lin28A^{S-} CICs exhibit significantly higher frequency of diploid CICs, with a low percentage of polyploidy resulting from Trolox treatment in a dose dependent manner (FIG. 8e; *p<0.05). Cell size of Lin28A^{S-} CICs are smaller than Lin28A^{S+} CICs within each ploidy group and are consistent in size for each ploidy group, regardless of Trolox dosing treatment (FIG. 8f, *p<0.05).

### Discussion

The heterogeneous mixture of CICs includes fibroblasts, endothelial, and hematopoietic cells that serve a critical role in both homeostasis and in response to injury. Preservation of structural integrity and compensation from normal biological aging to maintain homeostasis comes at the price of decreased tissue compliance, impaired contractile reserve, and replacement fibrosis following accrual of lost myocytes. CICs contribute to regulation of the aging process through effects upon myocardial structure, interaction with cardiomyocytes, and ongoing contribution to cellular renewal throughout lifespan. Indeed, CIC activity mediates many phenotypic alterations of myocardial structure that typify the aging heart.⁵⁻⁷ Although the heart is characterized by barely perceptible myocyte turnover in adulthood, CIC proliferative capacity is retained throughout life even as senescence markers accumulate.^{8, 9} Understanding regulatory mechanisms influencing CIC proliferation, senescence and polyploidization provides essential insights for aging cardiomyopathy and reveal strategic interventional approaches to mitigate progression of structural and functional degeneration, perhaps even in favor of prolonging beneficial cellular replacement and tissue repair.

Biological aging impacts both the structural integrity and the functional capacity of the myocardium. Previous studies have shown Lin28 up-regulation in cardiomyocytes to increase susceptibility to apoptosis.²⁵ In these studies, Lin28A also increased in response to cardiac aging (FIG. 1). Molecular signaling studies have affiliated Lin28A activity with downstream targets of cell cycle regulators (Myc, Ras, cyclins, cyclin-dependent kinases, hmga2, and PI3K-Akt), ribosomal biogenesis, mTOR, and insulin-dependent glycolytic metabolism.²⁸ Cell cycle, among various processes, becomes de-regulated during aging.⁵⁻⁷ CIC activity mediates many phenotypic alterations of the aging heart,⁵⁻⁷ with this study identifying Lin28A^{S+} as a marker of oxidative stress leading to senescent markers in CICs that would promote the aging process.

The aging heart is particularly impacted by oxidative stress.^{52, 53} Supplemental O₂ therapy (hyperoxia) is widely used in critical and intensive care settings and associated with lung injury and higher risk for mortality.^{54, 55} Hyperoxia also induces cardiac inflammation, toxicity, and pathophysiology.^{56, 57} These reports prompted speculation that standard cell culture of 21% O₂ tension recapitulates hyperoxia stress upon CICs, since physiologic 02 levels are closer to 5% or 1% in hypoxic niches.⁵⁸ Intracellular ROS production is an indicator of oxidative stress and is associated with biological aging³⁸⁻⁴², polyploidy,⁵⁹ senescence and loss of functional competency.⁶⁰ Increased reactive oxygen species (ROS) activity in response to oxidative stress impacts myocardial aging³⁸⁻⁴² and polyploidy⁵⁹ undoubtedly negatively impacts myocardial biology, promoting senescence and loss of functional competency.⁶⁰ Consistent with these findings, our results show that oxidative stress, through culturing in 21% O₂, directly increases frequency of Lin28A^{S+} CICs in concert with senescence associated b-gal⁺ and increased cell size (FIG. 2). Beta-gal⁺ CICs are larger in size regardless of Lin28A^{S+}, with b-gal⁻ CICs being significantly smaller. To our knowledge, this is the first study identifying Lin28A^{S+} as a marker of oxidative stress. This finding corroborates our previous study demonstrating human cCICs exhibit increased frequency of senescence when culturing in 21% O₂.⁶¹ Results from this study also demonstrate that senescence markers may be dampened when cultured at physiologic 02 levels, replicated with 1% O₂.

Oxidative stress also influences ploidy of the CICs. Lin28A^{S+} correlated with increased cellular ploidy content, evident in CICs grown in 1% and 21% O₂. Lin28A^{S-} CICs were mostly diploid in 1% O₂ versus an even mix of diploid and tetraploid when grown in the 21% oxygen at passage 2 (FIG. 3). Serial passaging for 10 splits of CICs in 21% O₂ yields a polyploid population, whereas a consistent mixture of diploid and tetraplopid CICs persists in 1% O₂ (FIG. 4). Our findings establish CIC polyploidization in response to oxidative stress with additional correlations to increased Lin28A^{S+} and higher ploidy content.

Cardiomyocyte polyploidization occurs during development, aging, and end-stage cardiomyopathies in conjunction with oxidative stress.^{32, 62-65} Our group subsequently confirmed prior reports of cardiomyocyte polyploidzation, extending this concept to tetraploidization of murine cCIC population.¹⁰ Polyploidization of CIC remains poorly investigated, and results of the present study advance the field by showing chronic oxidative stress correlates with CIC polyploidization and Lin28A^{S+} (FIG. 3).

Single cell RNA Seq (scRNA Seq) offers unprecedented insights regarding cellular heterogeneity by assessment of nuanced transcriptome variation. scRNA analyses reveal upregulation of multiple gene families in Lin28A^{S+} CICs associated with DNA transcription and cellular response to stress, specifically oxidative stress (FIGS. 5, 6). Fresh isolate Lin28A^{S+} preferentially typifies resident CIC lineage of endothelial rather than fibroblast origin, whereas hematopoietic CICs of extra-cardiac origin are mostly Lin28A^{S+}. The convergence of Lin28A^{S+} with endothelial lineage is consistent with endothelial biology in microenvironments of increased cellular stresses including reactive oxygen species.⁶⁶

The cardioprotective role of anti-oxidant therapy via reduction of ROS and oxidative stress is well established,⁴⁸⁻⁵¹ consistent with blunting of CIC stress an lowering of Lin28A^{S+} using the anti-oxidant Trolox, a water-soluble analog of vitamin E that has been tested in vivo. Cardioprotective action of Trolox was reported with enhanced cardiac recovery after ischemia/reperfusion of adult rats⁵¹, and it is tempting to speculate that this protective action rests in part with biological effects upon CICs. Trolox prompted biological actions upon CICs exposed to oxidative stress including suppression of Lin28A^{S+}, b-gal⁺ and polyploidization (FIGS. 7, 8). Trolox treatment also correlates with increased proliferation, consistent with higher frequency of diploid CICs with high Trolox dosing. Biological effects of Trolox upon CICs titrated in a dose-dependent fashion highlighting “cause and effect” of the compound to inhibit phenotypic markers of stress and senescence. The cellular mechanism of Trolox as a therapeutic treatment for oxidative stress with salutary effects for CICs in vivo consequential to aging or pathological injury should be addressed in future studies.

The CIC population plays a critical role in the structure and function of the myocardium. Lin28A^{S+} expression provides a novel marker for identification of cellular aging and senescence. Exploiting Lin28A^{S+} expression could yield further insights regarding regulatory pathways responsible for CIC stress and senescence consequential to oxidative stress responses. The identification of Lin28A^{S+} as a marker associated with oxidative stress, senescence, and polyploidization may provide for new therapeutic strategies to maintain a youthful phenotype in the non-myocyte population. Surface markers acquired during cellular stress or aging such as Lin28A^{S+} are valuable for future investigations intended to either mitigate deterioration of these cells or target senolytic therapies for slowing progression or elimination of myocardial pathogenesis.

### FIGURE LEGENDS

FIG. 1. Lin28A is expressed in human and rodent cardiac tissue. a. Adult human cardiac tissue demonstrating Lin28A is endogenous to both cardiomyocytes and cardiac interstitial cells, as shown by immunohistochemistry stain. b. Protein quantity of Lin28A is significantly increased in aged normal heart tissue compared to neonatal cardiac tissue. c. Quantification of Lin28A, normalized to GAPDH, verifies aged human cardiac tissue has an increased amount of Lin28A protein compared to neonatal human cardiac tissue. d. Adult FVB mouse cardiac tissue demonstrates Lin28A is endogenous to cardiac interstitial cells, shown by immunohistochemistry stain. e. Surface Lin28A is equally present in freshly isolated male and female FVB adult isolate CICs.

FIG. 2. Oxidative stress promotes Lin28A^{S+} expression together with CIC senescence phenotype. a. AMNIS IMAGESTREAM™ flow cytometry was used to separate and count FVB Total CICs for b-gal (Ch02—green), Brightfield (Ch04—grey), Lin28A^{S} (Ch05—red), side scatter (Ch06—pink), organized as Lin28A^{S+}/b-gal⁺ (top left), b-gal⁺ (top right) Lin28A^{S-} (bottom left), Lin28A^{S-}/b-gal⁻ (bottom right). b. Fresh isolate CICs are mostly Lin28A^{S-}/b-gal⁻, compared to 1% and 21% O₂. c. The frequency of Lin28A^{S+} CICs is significantly increased in higher O₂ culture. d. The size of Lin28A^{S+} CICs is significantly larger compared to Lin28A^{S-} CICs, regardless of O₂ culture. e. The frequency of b-gal⁺ CICs is significantly increased in higher O₂ culture. f. The size of b-gal⁺ CICs is significantly larger compared to b-gal⁻ CICs, regardless of O₂ culture. g. Of Lin28A^{S+} CICs, b-gal⁺ is more likely present in 21% O₂ culture. h. Lin28A^{S+}/b-gal⁺ CICs are significantly larger in size, regardless of O₂ culture.

FIG. 3. Diploid content favored by low oxidative stress conditions tracks with CIC phenotypes of Lin28A^{S-} and small size. a. AMNIS IMAGESTREAM™ flow cytometry was used to separate and count FVB Total CICs for Dapi (Ch01—purple), Brightfield (Ch04—grey), Lin28A^{S} (Ch05—red), side scatter (Ch06—pink), organized as mononuclear diploid (top left), binucleated diploid (top right) mononuclear tetraploid (bottom left), binucleated tetraploid (bottom right). b. The frequency of Lin28A^{S+} CICs is significantly increased in higher O₂ culture. c. Lin28A^{S+} of Total CIC expression is correlated with increased polyploidization. d. Lin28A^{S+} CICs is correlated with increased polyploidization. e. Lin28A^{S-} is correlated with diploid CICs. f. The Total CIC population is significantly correlated with increased polyploidization in higher O₂ culture. g. Diploid CICs are consistently small in cell size, regardless of Lin28A^{S} or O₂ culture. h. Tetraploid CICs are of increased size, particularly when Lin28A^{S+}, regardless of O₂ culture. i. Tetraploid CICs are of increased size, particularly when Lin28A^{S+}, regardless of O₂ culture. j. Total CICs demonstrate binucleation and Lin28A^{S+}, as shown by immunohistochemistry. k. Zoom in region of interest demonstrating binucleation and Lin28A^{S+}. l. Binucleation increases in Lin28A^{S+}, compared to Lin28A^{S-} CICs, in 1% O₂. m. Binucleation increases in Lin28A^{S+}, compared to Lin28A^{S-} CICs, in 21% O₂.

FIG. 4. Polyploidization of CICs occurs in response to chronic oxidative stress. a. Diploid CICs are maintained over passaging when exposed to 1% O₂ culture. b. Diploid CIC frequency is decreased, with increased tetraploid frequency over passaging when exposed to 21% O₂ culture. c. Reactive oxygen species (ROS) is increased by passage 2 in CICs maintained in 21%, compared to 1%, O₂ culture. d. The ROS to reactive nitrogen species (RNS) is increased by passage 2 in CICs maintained in 21%, compared to 1%, O₂ culture.

FIG. 5. Surface Lin28A CICs are predominantly endothelial and hematopoietic lineage. UMAP projection of Cardiac Interstitial Lin28^{S+/-} color-coded according to a. unsupervised clustering of gene signatures and b. as derived from either surface presence of Lin28. c. Cell contributions of Lin28^{S+/-} normalized to input of each main cell type as shown in UMAP (panels a and b). d. Heatmap representing the differential expressed genes from Lin28^{S+/-} populations.

FIG. 6. Surface Lin28A CICs upregulate gene markers demonstrating a stress response. a. Gene ontology (GO) results from Gene Ontology analysis annotated by Biological Process. Circle diameter represents the gene ratio from the 551/742 DEGs being expressed in the Lin28^{S+/-} cells, while significance level is color-coded according to heatmap scale. Dotplot representing expression of gene targets of stress associated GO terms b. Regulation of DNA-templated transcription in response to stress, c. Regulation of stress-activated MAPK cascade, d. Regulation of transcription from RNA polymerase II promoter in response to stress, e. Response to endoplasmic reticulum stress and f. Cellular response to oxidative stress in both Lin28^{S+/-} populations. Circle diameter represents the percentage of cells expressing a particular gene, while normalized average expression is represented by color intensity.

FIG. 7. Oxidative stress-induced phenotypic changes in CICs inhibited by anti-oxidant treatment. a. The frequency of Lin28A^{S+} expression in Total CICs cultured in 21% O₂ is significantly decreased when treated with Trolox. b. The frequency of Bgal positive expression in Total CICs cultured in 21% O₂ is significantly decreased when treated with Trolox. c. The frequency of Lin28^{S+}/b-gal⁺ expression in Total CICs cultured in 21% O₂ is significantly decreased when treated with Trolox. d. The size of Lin28^{S+} CICs is reduced by Trolox treatment in a dose dependent manner. e. The size of Lin28^{S+}/b-gal⁺ CICs is reduced by Trolox treatment in a dose dependent manner. f. Proliferation rate is higher in Total CICs treated with Trolox in a dose dependent manner.

FIG. 8. Trolox increases diploid population and antagonizes conversion to higher ploidy. a. The frequency of diploid Total CICs cultured in 21% O₂ is significantly increased when treated with Trolox in a dose dependent manner. b. The frequency of Lin28^{S+} CICs and polyploidization in Total CICs cultured in 21% O₂ is significantly decreased when treated with Trolox in a dose dependent manner. c. The ploidy of Lin28^{S+} CICs cultured in 21% O₂ is unchanged with Trolox. d. The size of Lin28^{S+} CICs treated with Trolox in a dose dependent manner are significantly larger based on ploidy content. e. The frequency of diploid Lin28^{S-} CICs cultured in 21% O₂ is significantly increased when treated with Trolox in a dose dependent manner. f. The size of Lin28^{S-} CICs treated with Trolox in a dose dependent manner are relatively unchanged based on ploidy content.

### SUPPLEMENT FIGURE LEGENDS

FIG. 9A-B, or Supplement FIG. 1. Gating strategy on AMNIS™ for Lin28A^{S+/-} and b-galactosidase. a. AMNIS IMAGESTREAM™ flow cytometry gating sorted based on size, in focus, b-gal, Lin28, and measured for frequency and area. b. AMNIS IMAGESTREAM™ flow cytometry was used to separate and count FVB Total CICs for b-gal (Ch02—green), Brightfield (Ch04—grey), Lin28^{S+/-} (Ch05—red), side scatter (Ch06—pink), organized as Lin28A^{S+}/b-gal⁺ (top), b-gal⁺ (second to top) Lin28A^{S-} (third to top), Lin28A^{S-}/b-gal⁻ (bottom).

FIG. 10, or Supplement FIG. 2. Hydrogen peroxide increases frequency of Lin28A^{S+} CICs. Treatment with hydrogen peroxide used as a positive control to confirm Lin28A^{S+} increases from oxidative stress.

FIG. 11, or Supplement FIG. 3. Chronic oxidative stress increases markers of senescence.

a. Of Lin28A^{S-} CICs, b-gal⁺ is more likely present in 21% O₂ culture. b. Of Lin28A^{S-} CICs, b-gal⁺ CICs are significantly larger in size, regardless of 02 culture. c. Of b-gal⁺ CICs, Lin28A^{S+} is equally present in 1% and 21% O₂ culture. d. Of b-gal⁺ CICs, Lin28A^{S+} CICs are significantly larger in size, regardless of O₂ culture. e. Of b-gal⁻ CICs, nearly all cells are Lin28A^{S-} in fresh isolate, 1% and 21% O₂ culture. d. Of b-gal⁻ CICs, Lin28A^{S+} and Lin28A^{S-} CICs are approximately the same size, regardless of O₂ culture.

FIG. 12, or Supplement FIG. 4. Gating strategy on AMNIS™ for Lin28A^{S+/-} and ploidy. a. AMNIS IMAGESTREAM™ flow cytometry gating sorted based on size, in focus, Dapi, Lin28, ploidy and measured for frequency and area. b. AMNIS IMAGESTREAM™ flow cytometry was used to separate and count FVB Total CICs for Dapi (Ch01—purple), Brightfield (Ch04—grey), Lin28A^{S+/-} (Ch05—red), side scatter (Ch06—pink), organized as mononuclear diploid (top left set), mononuclear tetraploid (bottom left set) binuclear diploid (top right set), binuclear tetraploid (bottom right set).

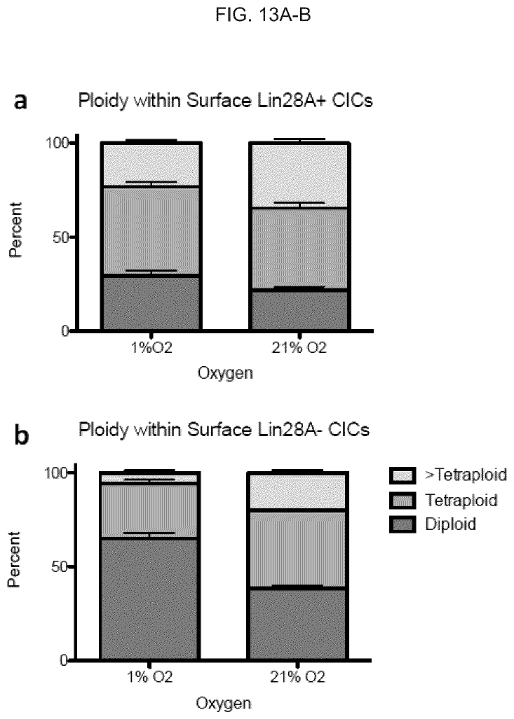

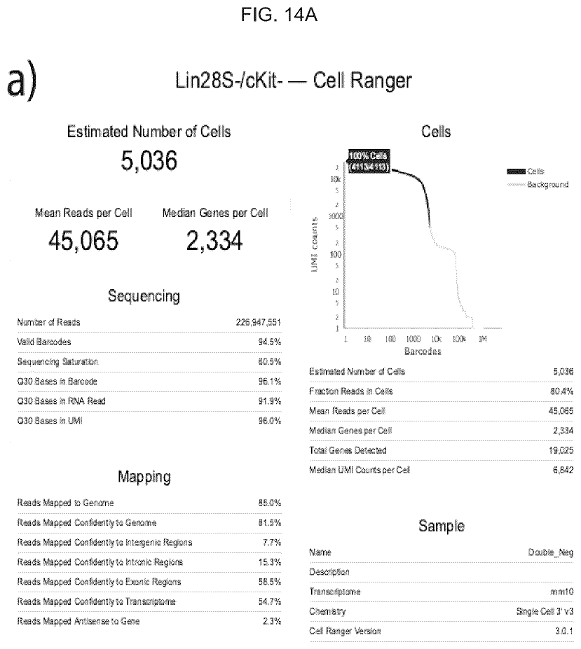

FIG. 13, or Supplement FIG. 5. Chronic oxidative stress increases polyploidization in both Lin28A^{S+/-}. a. The frequency of diploid Lin28A^{S+} CICs is greater in 1% O₂ compared to 21% O₂. b. The frequency of diploid Lin28A^{S-} CICs is greater in 1% O₂ compared to 21% O₂.

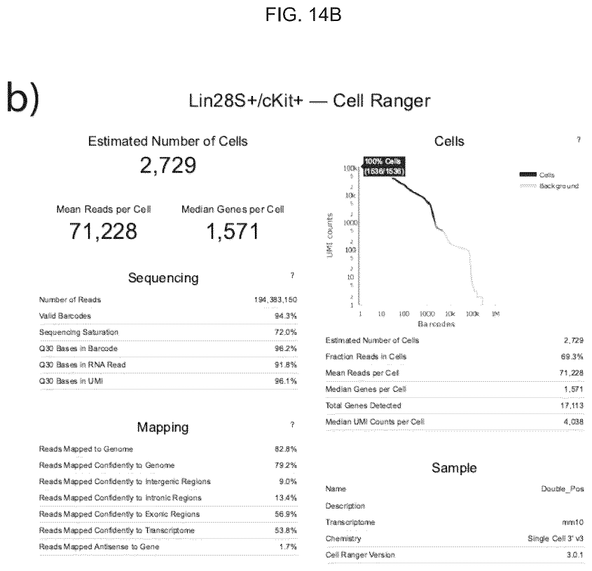

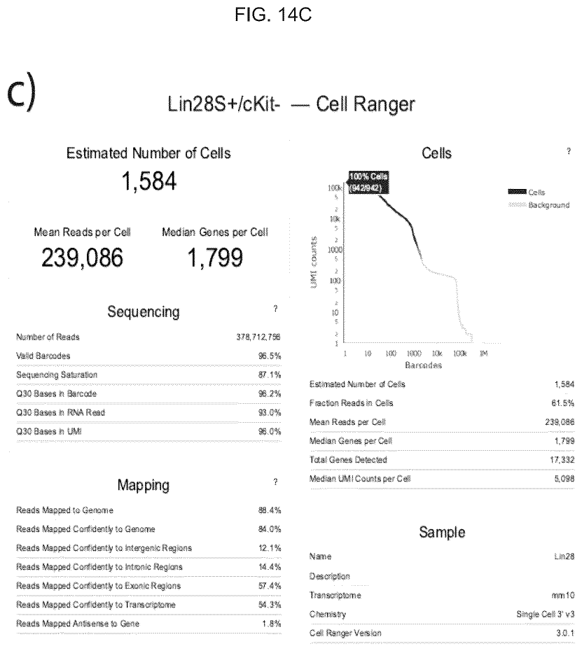

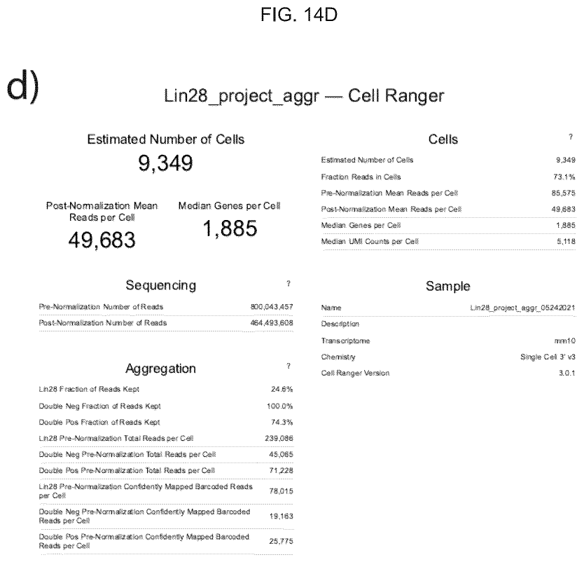

FIG. 14, or Supplement FIG. 6. Quality control for single cell RNA-SEQ experiment. CellRanger 3.0.1 quality control summary for a) Lin28S−/cKit−, b) Lin28S+/cKit+ and c) Lin28S+/cKit−. d) CellRanger 3.0.1 Library aggregation summary.

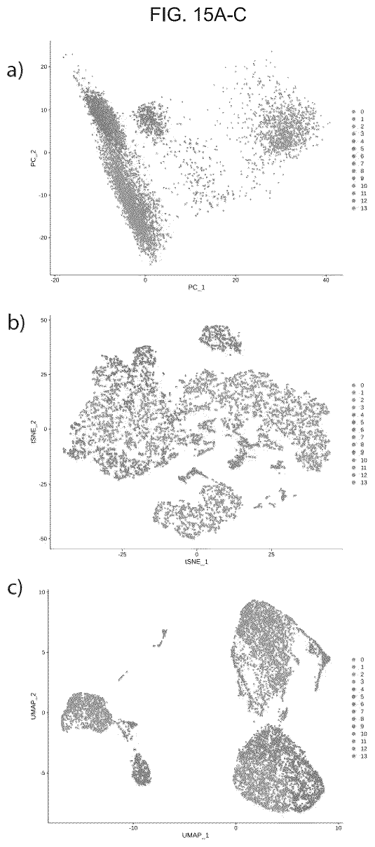

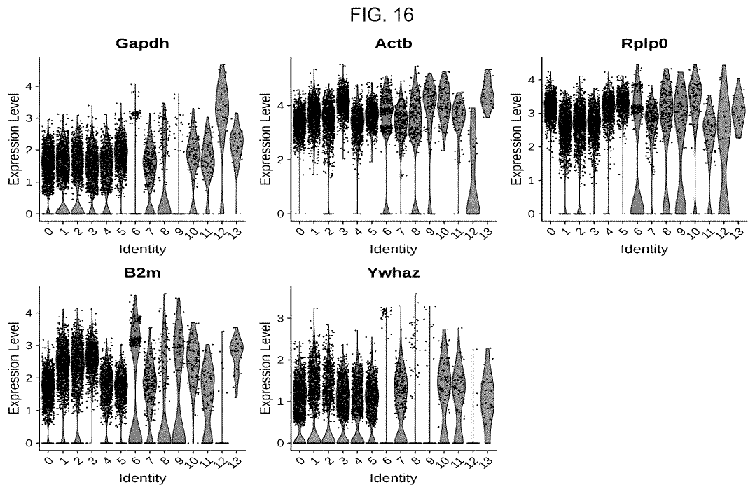

FIG. 15, or Supplement FIG. 7. Unsupervised scRNAseq clustering reveals 14 clusters. a) Principal component analysis (PCA), b) t-Distributed Stochastic Neighbor Embedding (t-SNE) and c) Uniform Manifold Approximation and Projection (UMAP) dimensionality reduction projections of single cell data color coded by detected unsupervised clusters.

FIG. 16, or Supplement FIG. 8. Unsupervised scRNAseq clusters validated via expression of house-keeping genes. Violin plots indicating expression of Gapdh, Actb, Rplp0, B2m and Ywhaz.

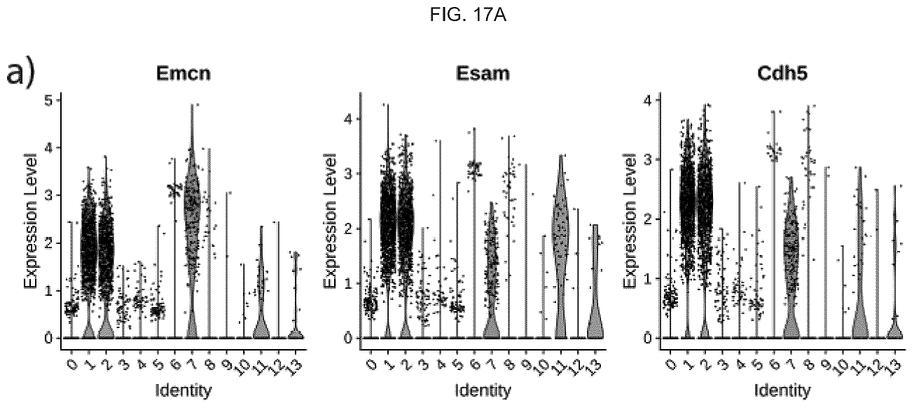

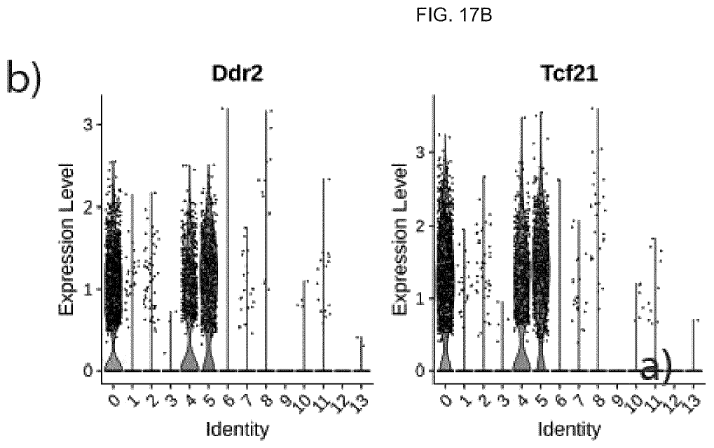

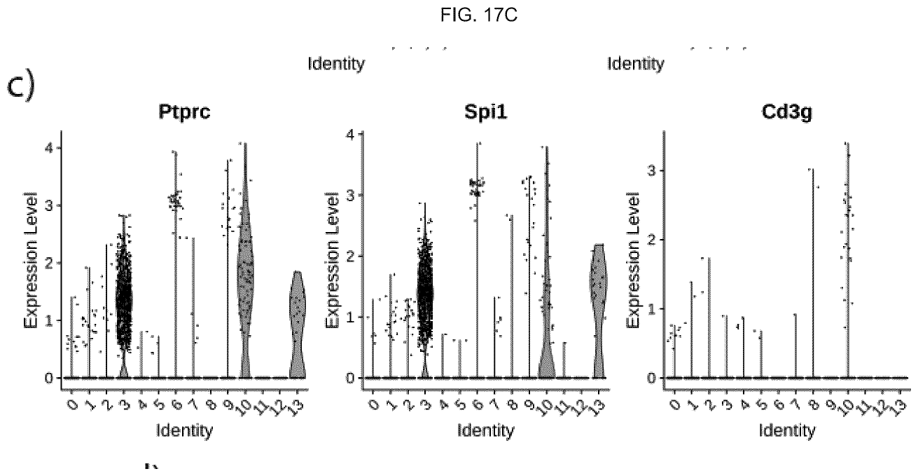

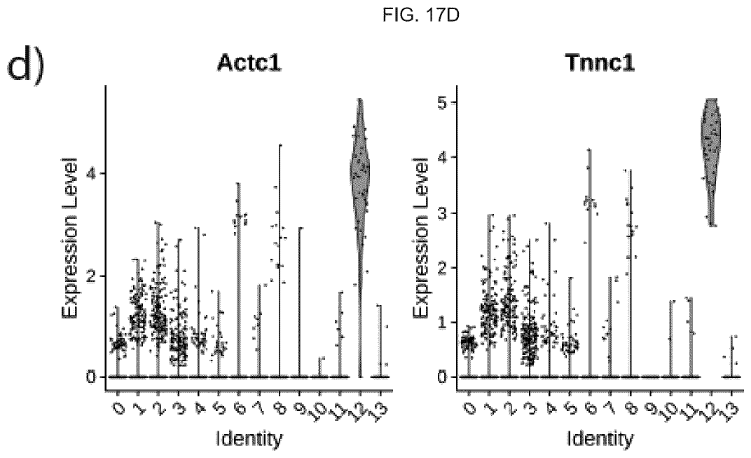

FIG. 17, or Supplement FIG. 9. Cell type annotation of unsupervised clusters via expression of canonical cell markers. Violin plots identifying a) Endothelial, b) Fibroblast, c) Hematopoietic and d) Myocyte cells based on expression of cell markers.

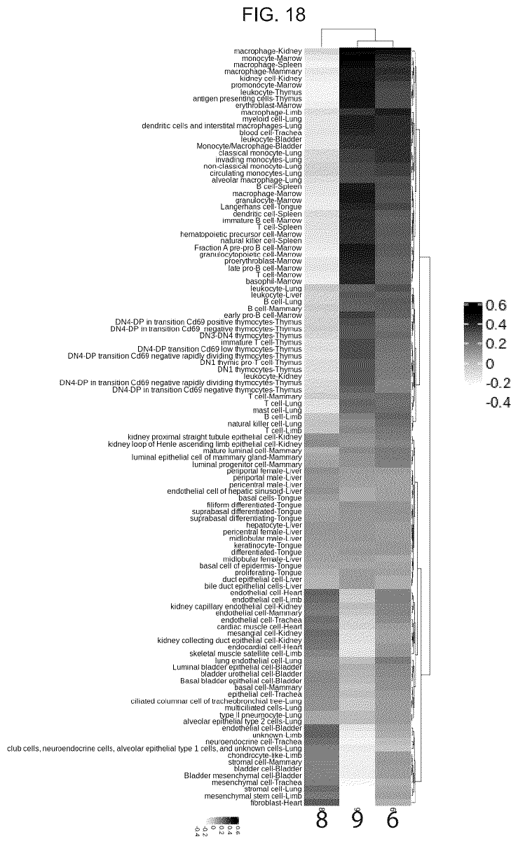

FIG. 18, or Supplement FIG. 10. Cell type annotation of unsupervised clusters 6, 8 and 9 via cross-reference to the Tabula muris cell atlas. Heatmap representing cell type correlation scores between the cluster expression matrix with the Tabula muris cell expression data.

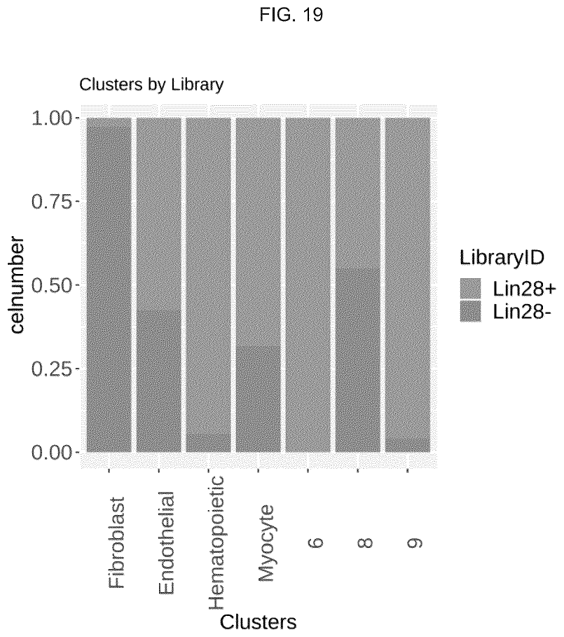

FIG. 19, or Supplement FIG. 11. Cell contributions of Lin28^{S+/-} normalized to input of each main cell type and cluster as shown in UMAP. (FIG. 1 panels a and b).

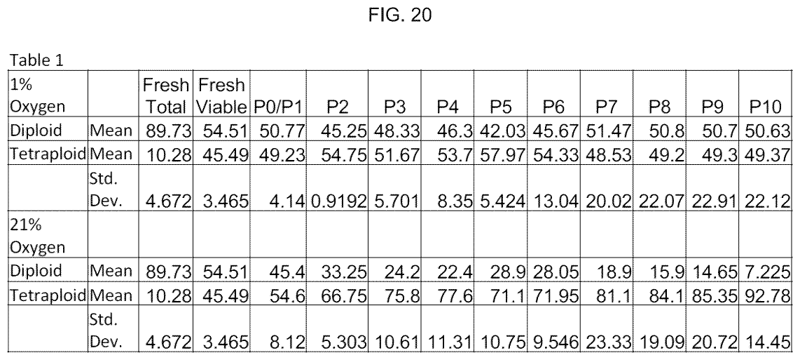

FIG. 20 illustrates Supplement Table 1. Polyploidization in CICs occurs in response to chronic oxidative stress. CICs demonstrate a shift from a diploid to a tetraploid state when cultured in 21% O₂.

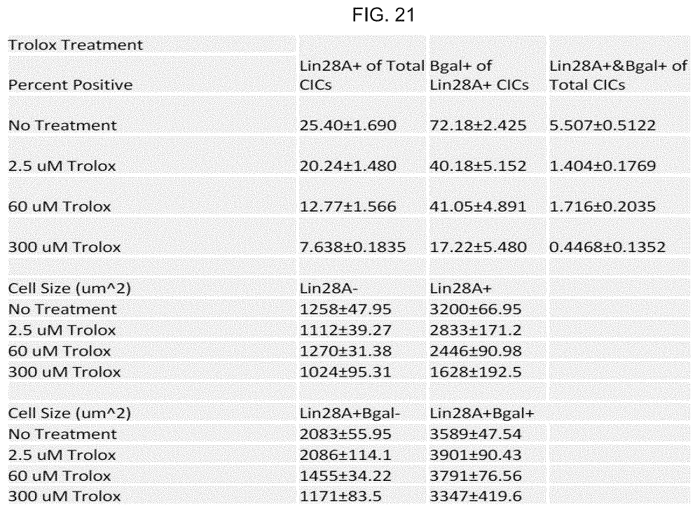

FIG. 21 illustrates Supplement Table 2. Trolox inhibits oxidative stress-induced phenotype changes in CICs. The frequency of Lin28A^{S+} CICs cultured in 21% O₂ is significantly decreased when treated with Trolox in a dose dependent manner.

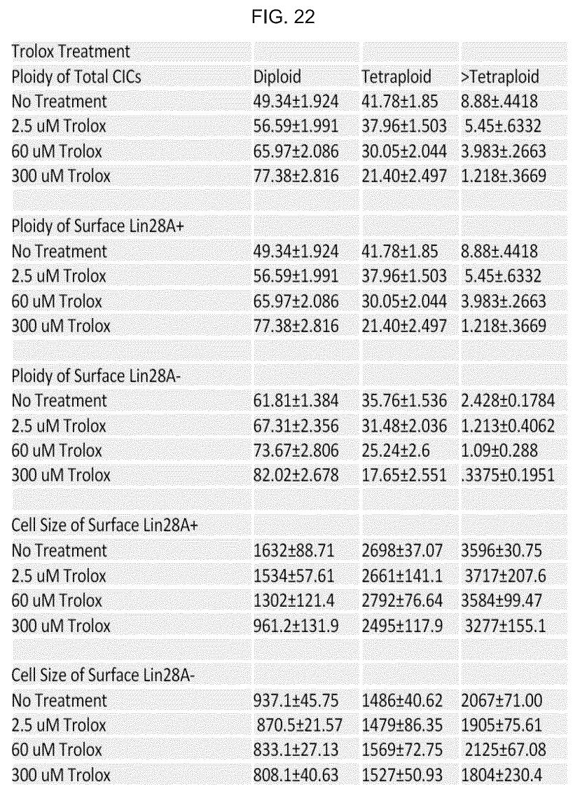

FIG. 22 illustrates Supplement Table 3. Trolox increases diploid population and antagonizes conversion to higher ploidy. The frequency of ploidy shifts in 21% O₂ cultured CICs is significantly decreased when treated with Trolox in a dose dependent manner.

### REFERENCES

1. Benjamin E J, et al, American Heart Association Council on E, Prevention Statistics C, Stroke Statistics S. Heart Disease and Stroke Statistics—2019 Update: A Report From the American Heart Association. Circulation 2019; 139:e56-e528.
2. Ko D T, et al. Life expectancy after an index hospitalization for patients with heart failure: a population-based study. Am Heart J 2008; 155:324-331.
3. Friedman B, et al. Medicare Hospital Stays: Comparisons Between the Fee-for-Service Plan and Alternative Plans, 2006: Statistical Brief #66.

Healthcare Cost and Utilization Project (HCUP) Statistical Briefs. Rockville (MD), 2006.
4. Writing Group M, Mozaffarian D, et al, American Heart Association Statistics C, Stroke Statistics S. Heart Disease and Stroke Statistics-2016 Update: A Report From the American Heart Association. Circulation 2016; 133:e38-360.
5. Gude N A, et al. Cardiac ageing: extrinsic and intrinsic factors in cellular renewal and senescence. Nat Rev Cardiol 2018; 15:523-542.
6. Biernacka A, et al. Aging Cardiac Fibrosis. Aging Dis 2011; 2:158-173.
7. Frangogiannis N G. Cardiac fibrosis: Cell biological mechanisms, molecular pathways and therapeutic opportunities. Mol Aspects Med 2019; 65:70-99.
8. Rota M, et al. Aging Effects on Cardiac Progenitor Cell Physiology. Compr Physiol 2015; 5:1775-1814.
9. Sanada F, et al. c-Kit-positive cardiac stem cells nested in hypoxic niches are activated by stem cell factor reversing the aging myopathy. Circ Res 2014; 114:41-55.
10. Broughton K M, et al. Cardiac interstitial tetraploid cells can escape replicative senescence in rodents but not large mammals. Commun Biol 2019; 2:205.
11. Shyh-Chang N, Daley G Q. Lin28: primal regulator of growth and metabolism in stem cells. Cell Stem Cell 2013; 12:395-406.
12. Hattori A, et al. Regulation of Stem Cell Self-Renewal and Oncogenesis by RNA-Binding Proteins. Adv Exp Med Biol 2016; 907:153-188.
13. Moss E G, Tang L. Conservation of the heterochronic regulator Lin-28, its developmental expression and microRNA complementary sites. Dev Biol 2003; 258:432-442.
14. Piskounova E, et al. Lin28A and Lin28B inhibit let-7 microRNA biogenesis by distinct mechanisms. Cell 2011; 147:1066-1079.
15. Rehfeld F, Rohde A M, Nguyen D T, Wulczyn F G. Lin28 and let-7: ancient milestones on the road from pluripotency to neurogenesis. Cell Tissue Res 2015; 359:145-160.
16. Thornton J E, Gregory R I. How does Lin28 let-7 control development and disease? Trends Cell Biol 2012; 22:474-482.
17. Tsialikas J, Romer-Seibert J. LIN28: roles and regulation in development and beyond. Development 2015; 142:2397-2404.
18. Bao M H, Feng X, Zhang Y W, Lou X Y, Cheng Y, Zhou H H. Let-7 in cardiovascular diseases, heart development and cardiovascular differentiation from stem cells. IntJMol Sci 2013; 14:23086-23102.
19. Li J, et al. Inhibition of the Let-7 Family MicroRNAs Induces Cardioprotection Against Ischemia-Reperfusion Injury in Diabetic Rats. Ann Thorac Surg 2016; 102:829-835.
20. Satoh M, et al. A cellular microRNA, let-7i, is a novel biomarker for clinical outcome in patients with dilated cardiomyopathy. J Card Fail 2011; 17:923-929.
21. Seeger T, et al. Inhibition of let-7 augments the recruitment of epicardial cells and improves cardiac function after myocardial infarction. J Mol Cell Cardiol 2016; 94:145-152.
22. Tolonen A M, et al. Inhibition of Let-7 microRNA attenuates myocardial remodeling and improves cardiac function postinfarction in mice. Pharmacol Res Perspect 2014; 2:e00056.
23. Aguirre A, et al. In vivo activation of a conserved microRNA program induces mammalian heart regeneration. Cell Stem Cell 2014; 15:589-604.
24. Du Y, et al. Let-7a regulates expression of beta1-adrenoceptors and forms a negative feedback circuit with the beta1-adrenoceptor signaling pathway in chronic ischemic heart failure. Oncotarget 2017; 8:8752-8764.
25. Joshi S, et al. A cardiac myocyte-restricted Lin28/let-7 regulatory axis promotes hypoxia-mediated apoptosis by inducing the AKT signaling suppressor PIK3IP1. Biochim Biophys Acta 2016; 1862:240-251.
26. Gray C, et al. Let-7 miRNA profiles are associated with the reversal of left ventricular hypertrophy and hypertension in adult male offspring from mothers undernourished during pregnancy after preweaning growth hormone treatment. Endocrinology 2014; 155:4808-4817.
27. Shi F, Yu W, Wang X. Bistable switch in let-7 miRNA biogenesis pathway involving Lin28. Int J Mol Sci 2014; 15:19119-19133.
28. Viswanathan S R, Daley G Q. Lin28: A microRNA regulator with a macro role. Cell 2010; 140:445-449.
29. Anatskaya O V, Vinogradov A E. Genome multiplication as adaptation to tissue survival: evidence from gene expression in mammalian heart and liver. Genomics 2007; 89:70-80.
30. Silva I S, et al. Polyploidy and nuclear phenotype characteristics of cardiomyocytes from diabetic adult and normoglycemic aged mice. Acta Histochem 2018; 120:84-94.
31. Herget G W, et al. DNA content, ploidy level and number of nuclei in the human heart after myocardial infarction. Cardiovasc Res 1997; 36:45-51.
32. Yan S M, et al. Changes in DNA content of myocardial cells after cardiac explantation. Adv Clin Path 1999; 3:23-27.
33. Rivello H G, et al. Cardiac myocyte nuclear size and ploidy status decrease after mechanical support. Cardiovasc Pathol 2001; 10:53-57.
34. McCrann D J, et al. Vascular smooth muscle cell polyploidy: an adaptive or maladaptive response? J Cell Physiol 2008; 215:588-592.
35. Yang D, et al. Increased polyploidy in aortic vascular smooth muscle cells during aging is marked by cellular senescence. Aging Cell 2007; 6:257-260.
36. Gray G A, et al. Resident cells of the myocardium: more than spectators in cardiac injury, repair and regeneration. Curr Opin Physiol 2018; 1:46-51.
37. Feng J, et al, Non-Cardiomyocytes in Heart Regeneration. Curr Drug Targets 2018; 19:1077-1086.
38. Li S Y, et al. Aging induces cardiac diastolic dysfunction, oxidative stress, accumulation of advanced glycation end products and protein modification. Aging Cell 2005; 4:57-64.
39. Cesselli D, et al. Cardiac Cell Senescence and Redox Signaling. Front Cardiovasc Med 2017; 4:38.
40. Dai D F, et al. Cardiac aging: from molecular mechanisms to significance in human health and disease. Antioxid Redox Signal 2012; 16:1492-1526.
41. Dai D F, et al. Mitochondrial oxidative stress in aging and healthspan. Longev Healthspan 2014; 3:6.
42. Di Lisa F, Bernardi P. Mitochondrial function and myocardial aging.

A critical analysis of the role of permeability transition. Cardiovasc Res 2005; 66:222-232.
43. Macosko E Z, et al. Highly Parallel Genome-wide Expression Profiling of Individual Cells Using Nanoliter Droplets. Cell 2015; 161:1202-1214.
44. Fu R, et al. clustifyr: an R package for automated single-cell RNA sequencing cluster classification. F1000Res 2020; 9:223.
45. Schaum C E, Buckling A, Smirnoff N, Studholme D J, Yvon-Durocher G. Environmental fluctuations accelerate molecular evolution of thermal tolerance in a marine diatom. Nat Commun 2018; 9:1719.
46. Yu G, et al, clusterProfiler: an R package for comparing biological themes among gene clusters. OMICS 2012; 16:284-287.
47. Tabula Muris C, Overall c, Logistical c, Organ c, processing, Library p, sequencing, Computational data a, Cell type a, Writing g, Supplemental text writing g, Principal i. Single-cell transcriptomics of 20 mouse organs creates a Tabula Muris. Nature 2018; 562:367-372.
48. Besse S, et al. Antioxidant treatment prevents cardiac protein oxidation after ischemia-reperfusion and improves myocardial function and coronary perfusion in senescent hearts. J Physiol Pharmacol 2006; 57:541-552.
49. Costanzo S, et al., Polyphemus Observational Study I. Postoperative atrial fibrillation and total dietary antioxidant capacity in patients undergoing cardiac surgery: The Polyphemus Observational Study. J Thorac Cardiovasc Surg 2015; 149:1175-1182 e1171.
50. Molyneux C A, et al. Oxidative stress and cardiac microvascular structure in ischemia and reperfusion: the protective effect of antioxidant vitamins. Microvasc Res 2002; 64:265-277.
51. Sagach V F, et al. The water-soluble vitamin E analogue Trolox protects against ischaemia/reperfusion damage in vitro and ex vivo. A comparison with vitamin E. Pharmacol Res 2002; 45:435-439.
52. Liguori I, et al. Oxidative stress, aging, and diseases. Clin Interv Aging 2018; 13:757-772.
53. Martin-Fernandez B, Gredilla R. Mitochondria and oxidative stress in heart aging. Age (Dordr) 2016; 38:225-238.
54. Janz D R, Hollenbeck R D, Pollock J S, McPherson J A, Rice T W.

Hyperoxia is associated with increased mortality in patients treated with mild therapeutic hypothermia after sudden cardiac arrest. Crit Care Med 2012; 40:3135-3139.
55. Kilgannon J H, et al, Circulation 2011; 123:2717-2722.
56. Hafner C, et al. Hyperoxia Induces Inflammation and Cytotoxicity in Human Adult Cardiac Myocytes. Shock 2017; 47:436-444.
57. Rodgers J L, et al. Impact of hyperoxia on cardiac pathophysiology. J Cell Physiol 2019; 234:12595-12603.
58. Wenger R H, et al. Frequently asked questions in hypoxia research. Hypoxia (Auckl) 2015; 3:35-43.
59. Craige S E, Keaney J F, Jr. Polyploidy and dysregulated ROS signaling: the school of hard Nox? Cell Cycle 2009; 8:797.
60. Cianflone E, et al. Adult Cardiac Stem Cell Aging: A Reversible Stochastic Phenomenon? Oxid Med Cell Longev 2019; 2019:5813147.
61. Korski K I, et al. Hypoxia Prevents Mitochondrial Dysfunction and Senescence in Human c-Kit(+) Cardiac Progenitor Cells. Stem Cells 2019; 37:555-567.
62. Senyo S E, et al. Mammalian heart renewal by pre-existing cardiomyocytes. Nature 2013; 493:433-436.
63. Walsh S, et al. Cardiomyocyte cell cycle control and growth estimation in vivo—an analysis based on cardiomyocyte nuclei. Cardiovasc Res 2010; 86:365-373.
64. Bensley J G, et al. Three-dimensional direct measurement of cardiomyocyte volume, nuclearity, and ploidy in thick histological sections. Sci Rep 2016; 6:23756.
65. Sukhacheva T V, et al., Age-Related Features of Cardiomyocyte Ploidy in Patients with Hypertrophic Obstructive Cardiomyopathy. Bull Exp Biol Med 2015; 159:95-99.
66. Chang X, et al. Oxid Med Cell Longev 2021; 2021:6620677.

Although the invention has been described in the context of certain embodiments, it is intended that the patent will not be limited to those embodiments; rather, the scope of this patent shall encompass the full lawful scope of the appended claims, and lawful equivalents thereof.

1: A method for:
removing cardiac cells impaired or functionally diminished by stress or age,
initiating, inducing or accelerating a cardiac muscle repair or tissue regeneration, a cardiac vasculature repair or tissue regeneration or a cardiac connective tissue repair or tissue regeneration,
treating or ameliorating age-related or stress-related cardiomyopathy, and/or
treating or ameliorating a heart injury or dysfunction from 1 minute to 12 hours after, or immediately after, a myocardial infarction (MI),
comprising:
(a) (i) providing: a compound or composition capable of inhibiting or decreasing the activity of Lin28+, or ablating or killing a Lin28+-expressing cardiac cell,
wherein the compound or composition comprises:
(1) a polypeptide composition capable of inhibiting or decreasing the activity of Lin28+, or ablating or killing a Lin28+-expressing cardiac cell,
(2) a nucleic acid composition that is capable of inhibiting or decreasing the activity of Lin28+, or ablating or killing a Lin28+-expressing cardiac cell; or
(3) a compound capable of inhibiting or decreasing the activity of Lin28+, or ablating or killing a Lin28+-expressing cardiac cell,
and
(ii) introducing into, onto or approximate to (close to) the mammalian heart, or cardiac or heart tissue, or heart muscle, or cardiac vasculature or connective tissue: the compound or composition, or
(b) administering a pharmaceutically acceptable formulation or a pharmaceutical composition to a subject in need thereof,
wherein the pharmaceutically acceptable formulation or pharmaceutical composition is comprised of:
(1) a polypeptide composition capable of inhibiting or decreasing the activity of Lin28+, or ablating or killing a Lin28+-expressing cardiac cell,
(2) a nucleic acid composition that is capable of inhibiting or decreasing the activity of Lin28+, or ablating or killing a Lin28+-expressing cardiac cell; or
(3) a compound capable of inhibiting or decreasing the activity of Lin28+, or ablating or killing a Lin28+-expressing cardiac cell,
thereby
removing cardiac cells impaired or functionally diminished by stress or age,
initiating, inducing or accelerating cardiac muscle repair or tissue regeneration, cardiac vasculature repair or tissue regeneration or cardiac connective tissue repair or tissue regeneration,
treating or ameliorating age-related or stress-related cardiomyopathy, and/or
treating or ameliorating the heart injury or dysfunction, an injury subsequent to (or following, or optionally from 1 minute to 12 hours after, or immediately after) a myocardial infarction (MI).

2: The method of claim 1, wherein the anti-Lin28+ antibody carries or is conjugated to a cytotoxic payload.

3: The method of claim 1, wherein the nucleic acid composition that is capable of inhibiting or decreasing the activity of Lin28+, or ablating or killing a Lin28+-expressing cardiac cell is or comprises an inhibitory nucleic acid.

4: The method of claim 1, wherein the compound capable of inhibiting or decreasing the activity of Lin28+, or ablating or killing a Lin28+-expressing cardiac cell comprises: a small molecule, a lipid, a saccharide or a polysaccharide.

5: The method of claim 4, wherein the compound comprises: CL285032; TPEN, LI171, 6-hydroxy-DL-DOPA; or N-methyl-N-[3-(3-methyl[1,2,4]triazolo[4,3-b]pyridazin-6-yl)phenyl]acetamide; or, an enantiomer or stereoisomer thereof.

6: The method of claim 1, wherein polypeptide, the nucleic acid and/or the compound is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally.

7: The method of claim 1, wherein the nucleic acid comprises or is contained in a nucleic acid construct or a chimeric or a recombinant nucleic acid, or an expression cassette, vector, plasmid, phagemid or artificial chromosome, optionally stably integrated into the cell's chromosome, or optionally stably episomally expressed, and optionally the cell is a cardiac cell.

8: A kit comprising a compound, nucleic acid or polypeptide, or a formulation or a pharmaceutical composition, as used in a method of claim 1.

9-10. (canceled)

11: The method of claim 1, wherein the cardiac cells impaired or functionally diminished have reduced proliferative capacity and/or polyploidy.

12: The method of claim 1, wherein the polypeptide composition is or is comprised of an anti-Lin28+ antibody.

13: The method of claim 12, wherein the anti-Lin28+ antibody is a monoclonal antibody.

14: The method of claim 2, wherein the cytotoxic payload comprises a cytotoxic compound or cytotoxic radionuclide.

15: The method of claim 14, wherein the cytotoxic compound comprises calicheamicin, duocarymycin, a ribosome-inactivating protein (RIP) or ricin.

16: The method of claim 2, wherein the radionuclide comprises astatine-211, yttrium-90, lutetium-177 or iodine-131.

17: The method of claim 2, wherein the cytotoxic payload is covalently linked to the anti-Lin28+ antibody by a linker compound.

18: The method of claim 17, wherein the linker compound comprises disuccinimidyl suberate (DSS)) or the cytotoxic payload is conjugated to the anti-Lin28+ antibody using a metal chelator.

19: The method of claim 17, wherein the linker compound comprises diethylenetriaminepentaacetic acid (DTPA) or 1,4,7,10-tetraazacyclododecane-1,4,7,10-tetraacetic acid (DOTA).

20: The method of claim 3, wherein the inhibitory nucleic acid comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme capable of capable of inhibiting or decreasing the expression or activity of a Lin28+ protein, transcript and/or gene.

21: The method of claim 6, wherein the formulation or pharmaceutical composition is contained in or carried in a nanoparticle, a particle, a micelle or a liposome or lipoplex, a polymersome, a polyplex or a dendrimer;

22: The method of claim 6, wherein the compound, polypeptide or the nucleic acid, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.
